(19) United States
(12) Patent Application Publication
(10) Pub. No.: US 2007/0105851 A1
(43) Pub. Date: May 10, 2007

(54) Indolyl-thienopyrazinone derivatives useful for treating hyperproliferative disorders and diseases associated with angiogenesis

(75) Inventors: James Cook, East Hampton, CT (US); Cheng Bi, West Haven, CT (US); Gaetan Ladouceur, Guilford, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporation, West Haven, CT (US)

(21) Appl. No.: 10/579,938

(22) PCT Filed: Nov. 4, 2004
(86) PCT No.: PCT/US04/37208

§ 371 Date: May 19, 2006

Related U.S. Application Data
(60) Provisional application No. 60/524,359, filed on Nov. 21, 2003.

(51) Int. Cl. A61K 31/541; A61K 31/5377; C07D 498/02; A61K 31/498

(52) U.S. Cl. 514/227.5; 514/234.2; 514/249; 544/60; 544/118; 544/350

(57) ABSTRACT

This invention relates to a compound of Formula (I): (I) and its use in treating hyper-proliferative disorders and diseases associated with angiogenesis.

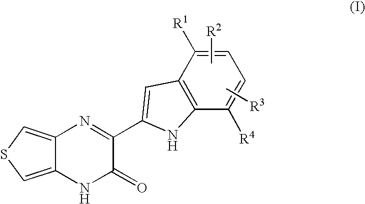

### FIELD OF THE INVENTION

This invention relates to novel indolyl-thienopyrazinone compounds, pharmaceutical compositions containing such compounds and the use of those compounds and compositions for the prevention and/or treatment of hyper-proliferative disorders and diseases associated with angiogenesis.

### DESCRIPTION OF THE INVENTION

Compounds of the Present Invention

One embodiment of this invention is a compound of Formula I 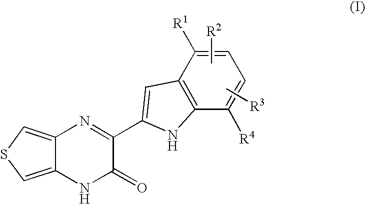
wherein
R¹ is selected from H, F, and Cl;
R² is selected from H, OH, CN, halo, C(O)R⁵, thienyl, pyrimidinyl, oxazolyl, furanyl,
   (C₁-C₃)alkyl, (C₂-C₆)alkenyl and (C₂-C₆)alkynyl, each optionally substituted with up to two substituents selected from OH, halo, and
      (C₁-C₃)alkoxy optionally substituted with (C₁-C₃)alkoxy,
   (C₁-C₆)alkoxy optionally substituted with (C₁-C₃)alkyl, (C₁-C₃)alkoxy, pyrrolidinyl, 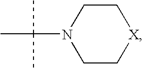
      and N[(C₁-C₃)alkyl]₂ where each alkyl group is independently optionally substituted with a substituent selected from (C₁-C₃)alkyl, (C₁-C₃)alkoxy OH, halo, 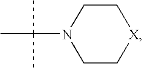
      and phenyl,
   N[(C₁-C₄)alkyl]₂ where each alkyl group is independently optionally substituted with up to two substituents independently selected from OH, (C₁-C₃)alkyl, halo, (C₁-C₃)alkoxy, and phenyl,
   pyridyl optionally substituted with up to two substituents independently selected from (C₁-C₃)alkyl, (C₁-C₃)alkoxy, and halo,
   phenyl optionally substituted with up to two substituents independently selected from (C₁-C₃)alkoxy, CN, halo, 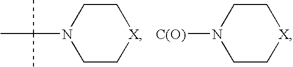
      C(O)N[(C₁-C₃)alkyl]₂ where each alkyl is optionally substituted with (C₁-C₃)alkoxy, and
      pyrrolidinyl optionally substituted with N[(C₁-C₃)alkyl]₂;
R³ is selected from H, halo, (C₁-C₃)alkyl, and (C₁-C₃)alkoxy;
R⁴ is selected from H, F, and Cl;
R⁵ is selected from OH, NHR⁶,
   N[(C₁-C₃)alkyl]R⁷ where said alkyl is optionally substituted with up to one substituent selected from (C₁-C₃)alkyl and (C₁-C₃)alkoxy,
   N[(C₁-C₃)alkyl]₂ where each alkyl is optionally substituted with up to two substituents independently selected from CN, OH, (C₁-C₃)alkoxy, N[(C₁-C₃)alkyl]₂, pyridyl, phenyl, S(O)₂(C₁-C₃)alkyl, tetrahydrofuryl, S(O)₂-phenyl, (C₃-C₆)cycloalkyl, and
      furyl optionally substituted with (C₁-C₃)alkyl,
   N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl, 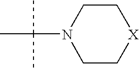
   optionally substituted with up to two substituents independently selected from N[(C₁-C₃)alkyl]₂, C(O)(C₁-C₃)alkyl, pyrrolidinyl, S(O)₂(C₁-C₃)alkyl, S(O)₂-phenyl, 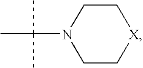
   oxo-dihydrobenzimidazolyl, pyrazinyl, C(O)NH₂, C(O)NH-phenyl, C(O)-furanyl, C(O)NH(C₁-C₃)alkyl,
      (C₁-C₃)alkyl optionally substituted with up to two substituents independently selected from OH, halo, (C₁-C₃)alkoxy, 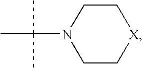
      pyrrolidinyl, C(O)-pyrrolidinyl, 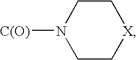
      and N[(C₁-C₃)alkyl]₂,
      phenyl optionally substituted with up to two substituents independently selected from (C₁-C₃)alkyl, (C₁-C₃)alkoxy, halo, CF₃, and CN, and
      pyridyl optionally substituted with (C₁-C₃)alkyl, CF₃, and CN, and
   pyrrolidinyl optionally substituted with up to two substituents independently selected from N[(C₁-C₄)alkyl]₂, C(O)NH₂, pyridyl, and
      (C₁-C₃)alkyl optionally substituted with up to two substituents independently selected from (C₁-C₃)alkoxy, and pyrrolidinyl;
R⁶ is selected from H,
   (C₁-C₄)alkyl optionally substituted with up to two substituents independently selected from OH, halo, (C₁-C₄)alkoxy, NHC(O)(C₁-C₃)alkyl, S—(C₁-C₃)alkyl, benzimidazolyl, thienyl, 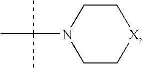
      N[(C₁-C₄)alkyl]₂ where each alkyl is independently optionally substituted with up to two substituents independently selected from OH, (C₁-C₃)alkoxy, halo, and phenyl,
      phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
      pyridyl optionally substituted up to two times with CF₃,
      indolyl optionally substituted up to two times with (C₁-C₃)alkyl,
      imidazolyl optionally substituted up to two times with (C₁-C₃)alkyl,
      furyl optionally substituted up to two times with (C₁-C₄)alkyl, and
      pyrrolidinyl optionally substituted with up to two substituents independently selected from (C₁-C₄)alkoxy, (O), and
         (C₁-C₄)alkyl optionally substituted with up to two substituents independently selected from OH, (C₁-C₃)alkoxy, and halo,
   indolyl optionally substituted up to two times with (C₁-C₃)alkyl,
   pyrazolyl optionally substituted with up to two substituents independently selected from (C₁-C₄)alkyl, (C₃-C₆)cycloalkyl, and
      phenyl optionally substituted with up to two substituents independently selected from (C₁-C₄)alkoxy, (C₁-C₄)alkyl, halo, CF₃, and CN,
   benzothiazolyl optionally substituted up to two times with (C₁-C₄)alkyl,
   thiazolyl optionally substituted up to two times with (C₁-C₄)alkyl,
   thiadiazolyl optionally substituted with up to two substituents independently selected from CF₃, (C₃-C₆)cycloalkyl, and (C₁-C₆)alkyl,
      phenyl optionally substituted with up to two substituents independently selected from CN, halo, CF₃, N[(C₁-C₄)alkyl]₂, indolyl, 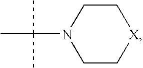
   (C₁-C₄)alkoxy, O-pyridyl optionally substituted with C(O)NH(C₁-C₄)alkyl,
      (C₁-C₄)alkyl optionally substituted with up to two substituents independently selected from pyridyl, OH, halo, and phenyl, and 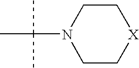
      optionally substituted with up to two substituents independently selected from (C₁-C₃)alkyl, and (C₁-C₄)alkoxy,
   pyridyl optionally substituted with phenoxy where said phenoxy is optionally substituted with up to two substituents independently selected from (C₁-C₄)alkyl and (C₁-C₄)alkoxy, and
   indazolyl optionally substituted up to two times with (C₁-C₄)alkyl;
R⁷ is selected from (C₁-C₃)alkoxy, pyrrolidinyl, tetrahydropyranyl,
   pyridyl optionally substituted with up to two substituents independently selected from (C₁-C₄)alkyl and (C₁-C₃)alkoxy,
   pyranyl optionally substituted with up to two substituents independently selected from (C₁-C₄)alkyl and (C₁-C₃)alkoxy,
   piperidinyl optionally substituted with up to two substituents independently selected from (C₁-C₃)alkyl, and (C₁-C₃)alkoxy, and
   phenyl optionally substituted with up to two substituents independently selected from (C₁-C₃)alkoxy, and (C₁-C₃)alkyl; and
X is selected from O, S, CH₂ and NH;
with the proviso that when R¹ is F or Cl, then R⁴ must be H, and when R⁴ is F or Cl, then R¹ must be H;
   or a pharmaceutically acceptable salt thereof.

The terms identified above have the following meaning throughout:

R² is attached to the indolyl moiety of the core molecule at either the 5 or 6 atom of the indolyl moiety.

R³ is attached to the core molecule at the 5 or 6 atom on the indolyl moiety that is not occupied by the R² group. That is, when R² is attached to the 5 atom of the indolyl moiety, then R³ is attached to the 6 atom of the indolyl moiety, and visa versa.

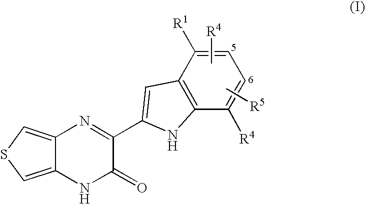

The term “optionally substituted” means that, unless indicated otherwise, the moiety so modified may have from one to up to at least two of the substituents indicated. Each substituent may replace any H atom on the moiety so modified as long as the replacement is chemically possible and chemically stable. For example, a chemically unstable compound would be one where each of two substituents is bonded to a single C atom through each substituent's heteroatom. Another example of a chemically unstable compound would be one where an alkoxy group is bonded to the unsaturated carbon of an alkene to form an enol ether. When there are two substituents on any moiety, each substituent is chosen independently of the other substituent so that, accordingly, the substituents can be the same or different.

The terms “(C₁-C₃)alkyl”, “(C₁-C₄)alkyl” and “(C₁-C₆)alkyl” mean linear or branched saturated carbon groups having from about 1 to about 3, about 4, or about 6 carbon atoms, respectively. Such groups include but are not limited to methyl, ethyl, n-propyl, isopropyl, and the like.

The term “(C₃-C₆)cycloalkyl” means a saturated monocyclic alkyl group of from 3 to about 6 carbon atoms and includes such groups as cyclopropyl, cyclobutyl, cyclopentyl, cyclohexyl.

The term “(C₂-C₆)alkenyl” means a linear or branched carbon group having from about 2 to about 6 C atoms wherein at least two adjacent C atoms in the alkenyl group are joined by a double bond, with the proviso that when a C atom is double bonded to one adjacent C atom, it must be single bonded to any other adjacent C atom. The alkenyl group is attached to the rest of the molecule through a single bond. Such groups include, ethenyl, allyl, isopropenyl, 2-butenyl, 2-ethyl-2-butenyl, 1-hexenyl and the like.

The term (C₂-C₆)alkynyl means a linear or branched carbon group having from about 2 to about 6 C atoms wherein at least two adjacent C atoms in the alkynyl group are joined by a triple bond, with the proviso that when a C atom is triple bonded to one adjacent C atom, it must be single bonded to any other adjacent C atom. The alkynyl group is attached to the rest of the molecule through a single bond. Such groups include, ethynyl, propargyl, 2-butynyl, 1-methyl-2-butynyl, 3-hexynyl and the like.

The terms “(C₁-C₃)alkoxy”, and “(C₁-C₄)alkoxy” means an alkyl group, as described above, bonded to an O atom. The O atom is the point of attachment of the (C₁-C₃)alkoxy group to the rest of the molecule. Such groups include but are not limited to methoxy, ethoxy, n-propoxy, isopropoxy, and the like.

The term “halo” means an atom selected from Cl, Br, and F.

The term “phenoxy” means a phenyl ring attached to an O atom, the O atom being attached to the rest of the molecule.

When “(O)” is used in a chemical formula, it means an O atom that is double bonded to the C or S atom to which it is attached.

The formula “N[C₁-C₃)alkyl]₂” means that each of the 2 possible alkyl groups attached to the N atom are selected independently from the other so that they may be the same or they may be different.

When a phenyl ring or a heterocycle is attached to the rest of the molecule, it is attached by replacing any H atom on the phenyl ring or on the heterocycle, respectively, with a bond to the rest of the molecule, as long as the replacement is chemically possible and chemically stable. 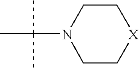
means morpholinyl, thiomorpholinyl, piperidinyl or piperazinyl. When X is NH, the 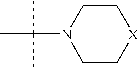
moiety is optionally substituted by replacing the H on the NH group with one of the desired substituents. When X is O, S, or CH₂, the 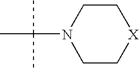
moiety is optionally substituted by replacing any H atom in the 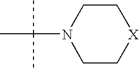
moiety with the desired substituent. When 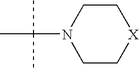
has two substituents, each is selected independently from the other so that they may be the same or different.

Representative compounds of the invention are shown by way of example in Table I.

TABLE 1

| | | | | Preparative |
|---|---|---|---|---|
| | | LCMS | LCMS | Method(s) |
| Example | | RT | Ion | (Example |
| Number | Structure | (min) | [M + H]⁺ | No.) |
| 1 | 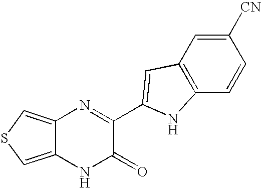 | 2.64 | 293.2 | 1 |
| 2 | 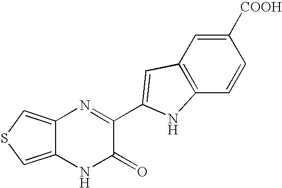 | 2.30 | 312.0 | 1, 2 |
| 3 | 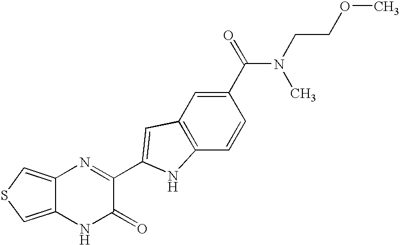 | 2.31 | 383.1 | 1, 2, 3 |
| 4 | 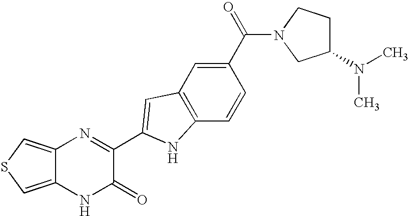 | 1.85 | 408.1 | 1, 2, 3 |
| 5 | 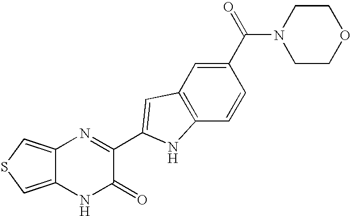 | 2.28 | 381.1 | 1, 2, 3 |
| 6 | 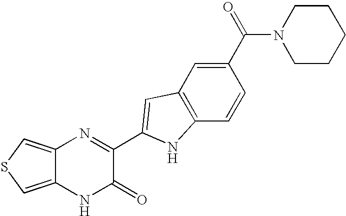 | 2.66 | 379.2 | 1, 2, 3 |
| 7 | 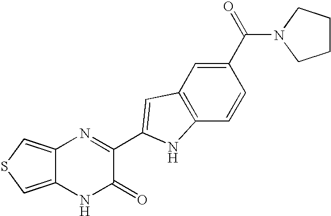 | 2.52 | 365.2 | 1, 2, 3 |
| 8 | 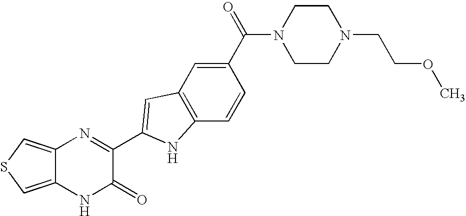 | 1.86 | 438.0 | 1, 2, 3 |
| 9 | 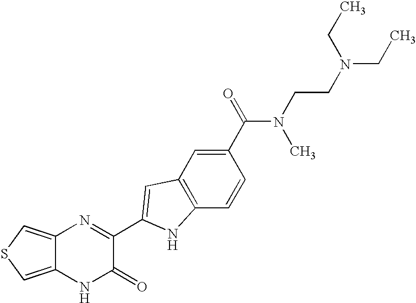 | 1.86 | 424.1 | 1, 2, 3 |
| 10 | 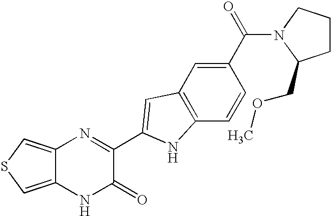 | 2.49 | 409.3 | 1, 2, 3 |
| 11 | 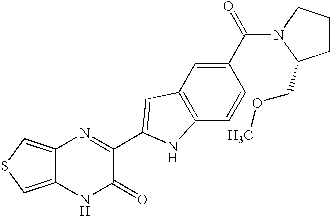 | 2.46 | 409.0 | 1, 2, 3 |
| 12 | 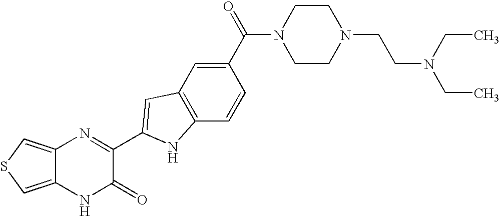 | 1.65 | 479.2 | 1, 2, 3 |
| 13 | 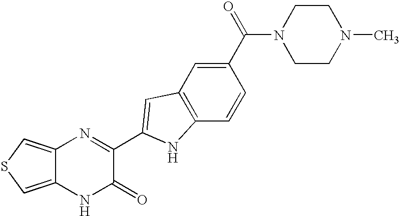 | 1.57 | 394.0 | 1, 2, 3 |
| 14 | 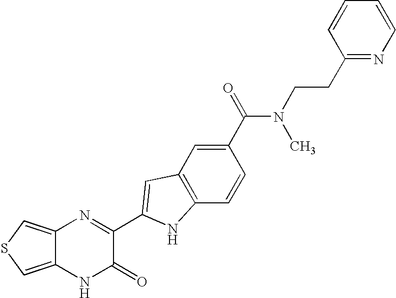 | 1.95 | 430.0 | 1, 2, 3 |
| 15 | 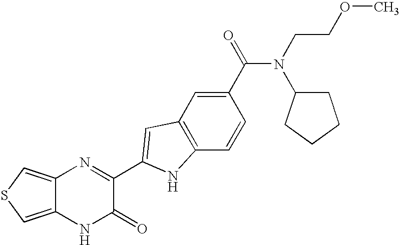 | 2.97 | 437.1 | 1, 2, 7, 3 |
| 16 | 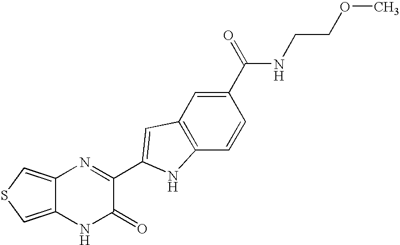 | 2.20 | 369.3 | 1, 2, 3 |
| 17 | 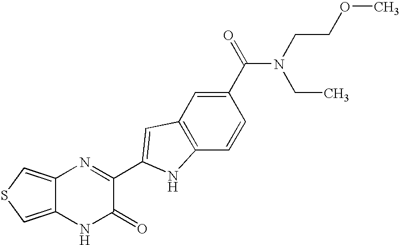 | 2.45 | 397.3 | 1, 2, 3 |
| 18 | 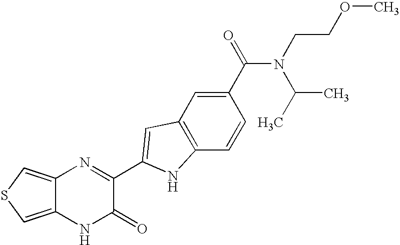 | 2.61 | 411.5 | 1, 2, 3 |
| 19 | 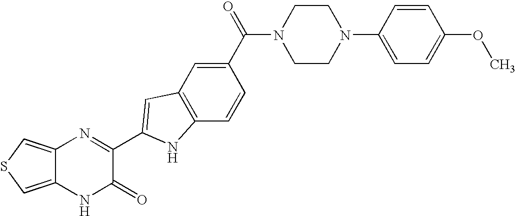 | 2.49 | 486.6 | 1, 2, 3 |
| 20 | 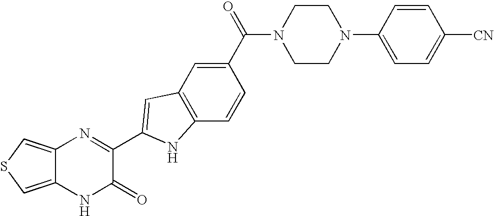 | 2.81 | 481.0 | 1, 2, 3 |
| 21 | 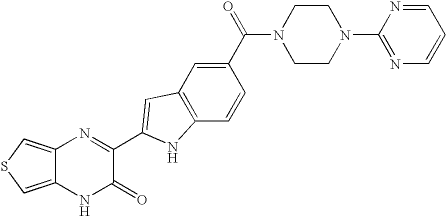 | 2.36 | 458.2 | 1, 2, 3 |
| 22 | 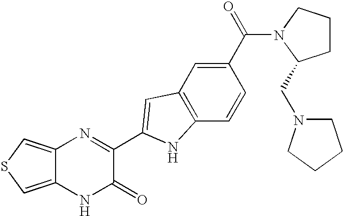 | 2.00 | 448.2 | 1, 2, 3 |
| 23 | 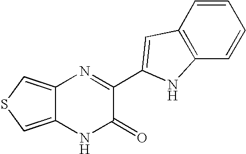 | | | 1, 2, 3 |
| 24 | 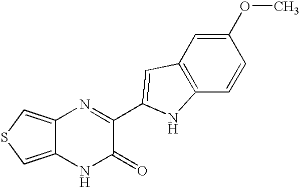 | | | 1 |
| 25 | 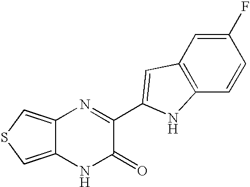 | | | 1 |
| 26 | 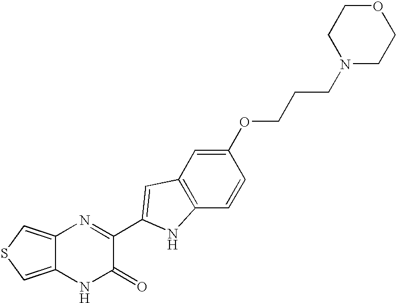 | | | 4, 1 |
| 27 | 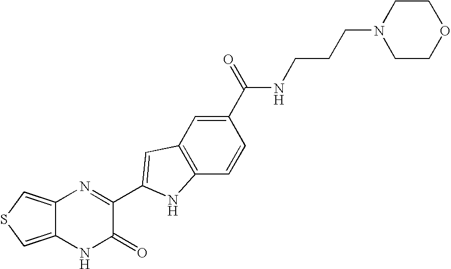 | | | 1, 2, 3 |
| 28 | 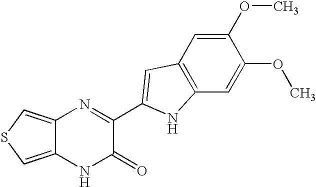 | | | 1, 2, 3 |
| 29 | 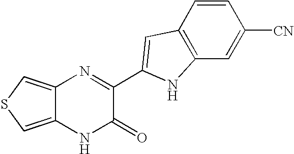 | | | 1 |
| 30 | 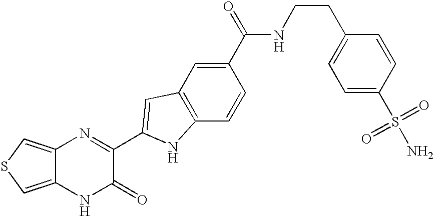 | | | 1, 2, 3 |
| 31 | 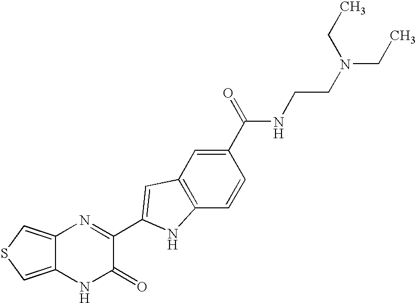 | | | 1, 2, 3 |
| 32 | 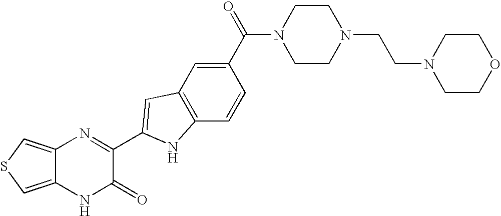 | | | 1, 2, 3 |
| 33 | 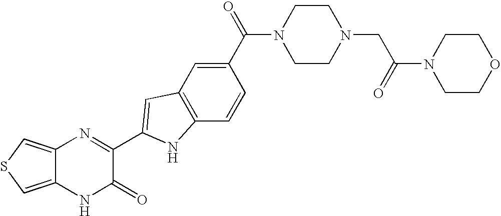 | | | 1, 2, 3 |
| 34 | 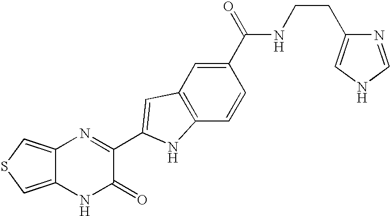 | | | 1, 2, 3 |
| 35 | 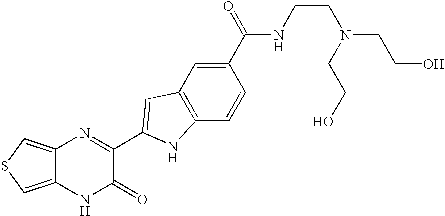 | | | 1, 2, 3 |
| 36 | 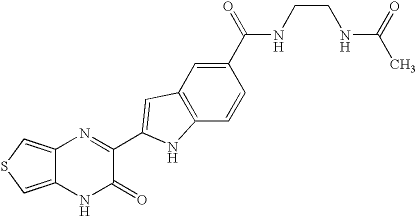 | | | 1, 2, 3 |
| 37 | 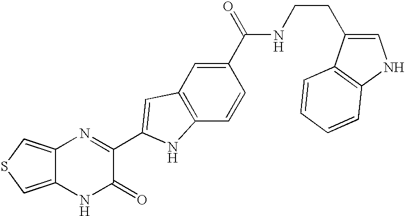 | | | 1, 2, 3 |
| 38 | 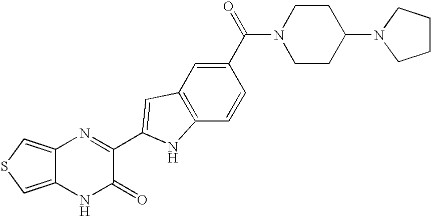 | | | 1, 2, 3 |
| 39 | 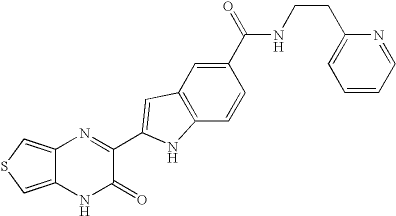 | | | 1, 2, 3 |
| 40 | 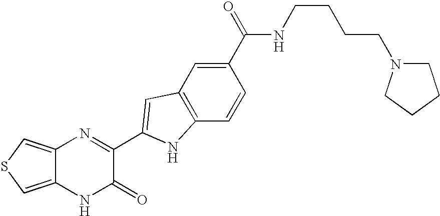 | | | 1, 2, 3 |
| 41 | 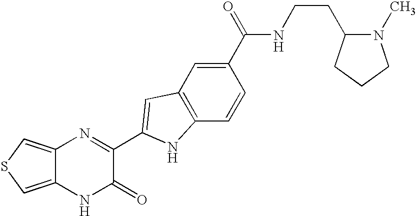 | | | 1, 2, 3 |
| 42 | 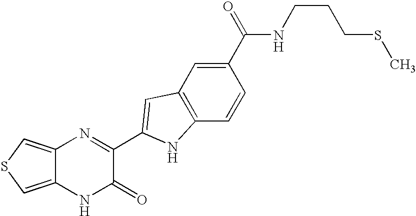 | | | 1, 2, 3 |
| 43 | 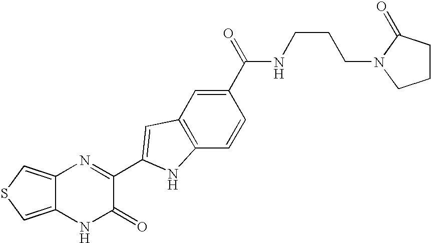 | | | 1, 2, 3 |
| 44 | 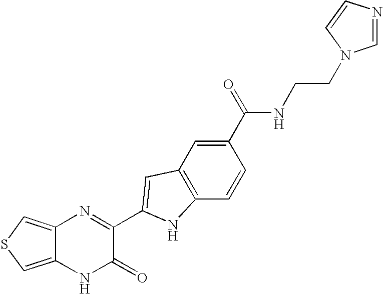 | | | 1, 2, 3 |
| 45 | 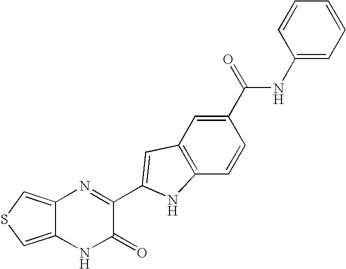 | | | 1, 2, 3 |
| 46 | 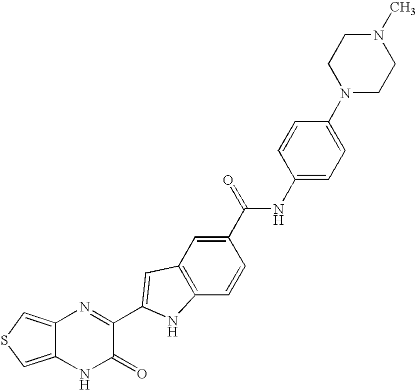 | | | 1, 2, 3 |
| 47 | 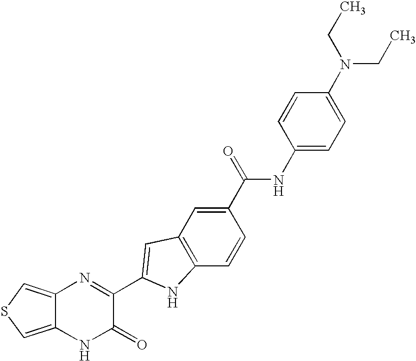 | | | 1, 2, 3 |
| 48 | 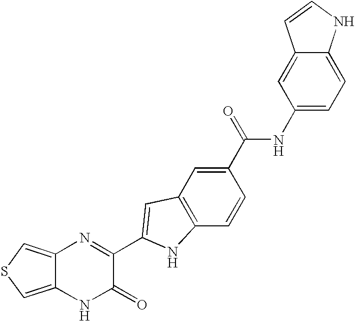 | | | 1, 2, 3 |
| 49 | 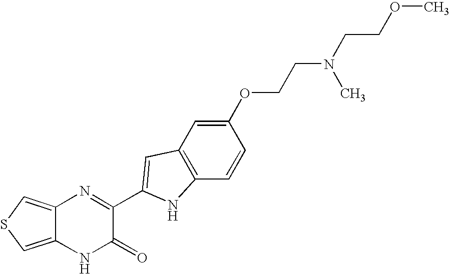 | | | 4, 1 |
| 50 | 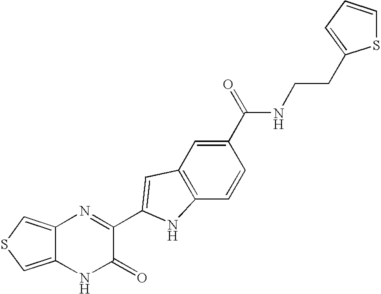 | | | 1, 2, 3 |
| 51 | 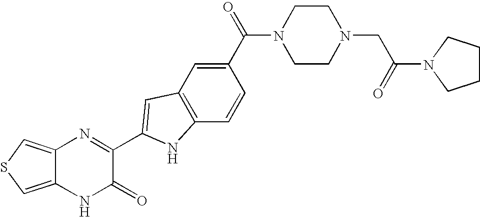 | | | 1, 2, 3 |
| 52 | 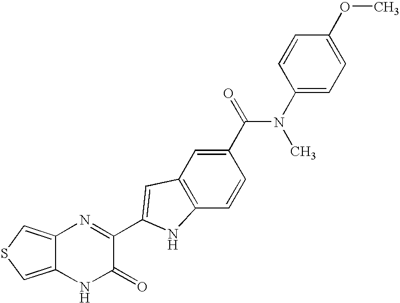 | | | 1, 2, 3 |
| 53 | 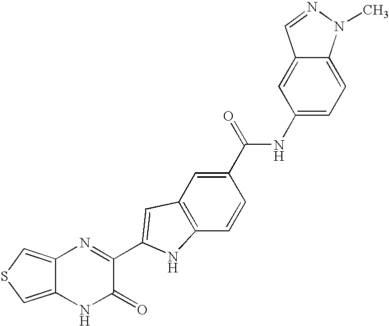 | | | 1, 2, 3 |
| 54 | 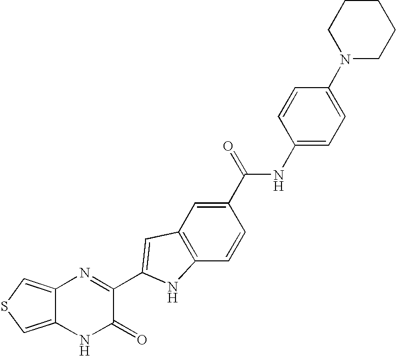 | | | 1, 2, 3 |
| 55 | 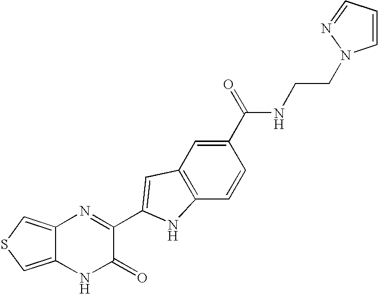 | | | 1, 2, 3 |
| 56 | 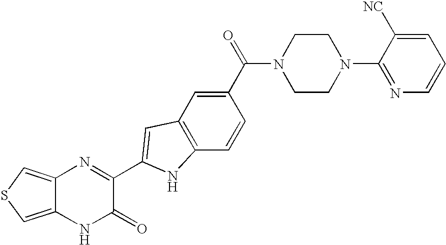 | | | 1, 2, 3 |
| 57 | 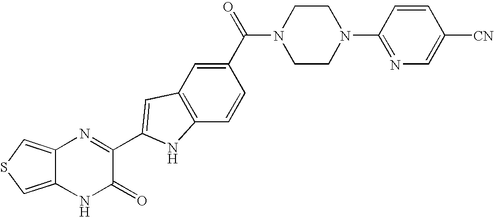 | | | 1, 2, 3 |
| 58 | 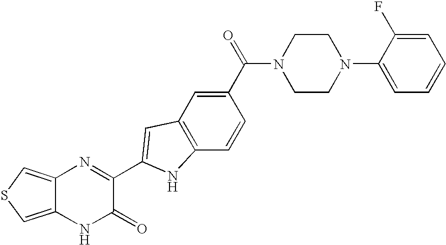 | | | 1, 2, 3 |
| 59 | 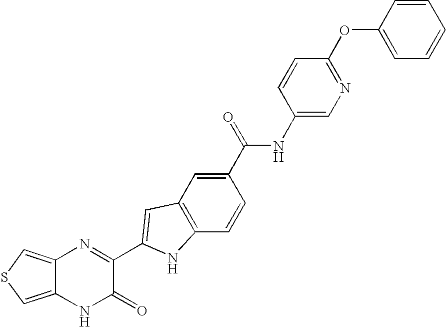 | | | 1, 2, 3 |
| 60 | 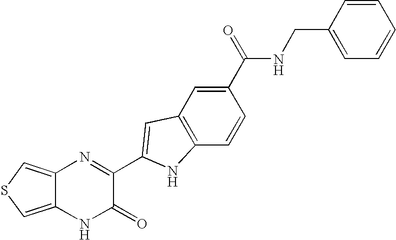 | | | 1, 2, 3 |
| 61 | 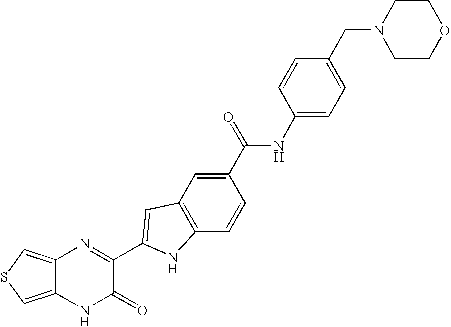 | | | 1, 2, 3 |
| 62 | 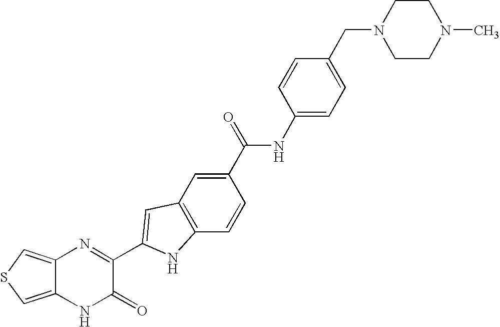 | | | 1, 2, 3 |
| 63 | 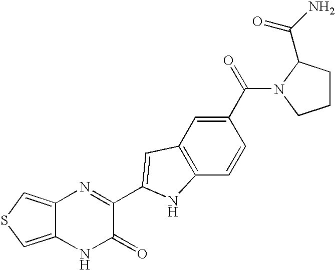 | | | 1, 2, 3 |
| 64 | 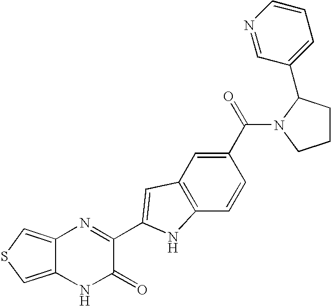 | | | 1, 2, 3 |
| 65 | 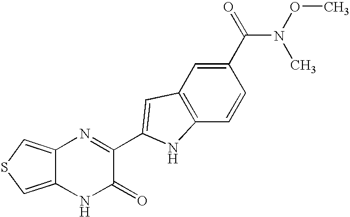 | | | 1, 2, 3 |
| 66 | 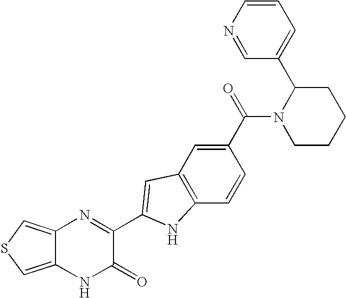 | | | 1, 2, 3 |
| 67 | 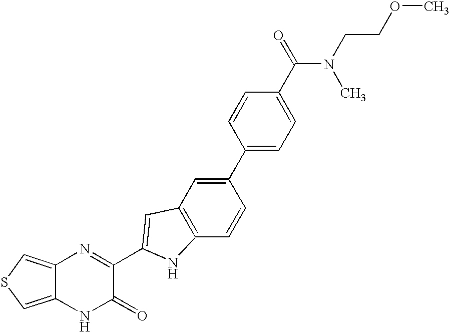 | | | 5, 1 (steps 2, 3), 2, 3 |
| 68 | 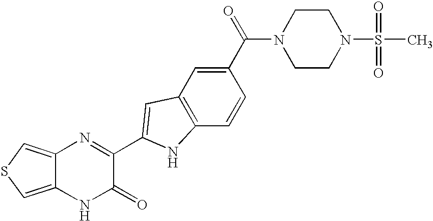 | | | 1, 2, 6, 3 |
| 69 | 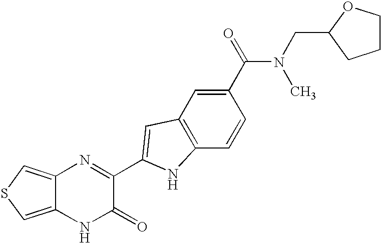 | | | 1, 2, 3 |
| 70 | 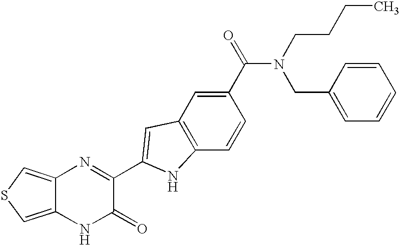 | | | 1, 2, 3 |
| 71 | 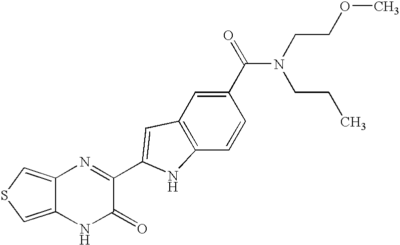 | | | 1, 2, 3 |
| 72 | 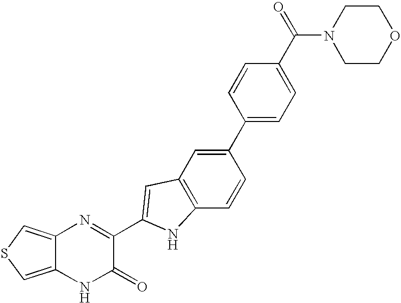 | | | 5, 1 (steps 2, 3), 2, 3 |
| 73 | 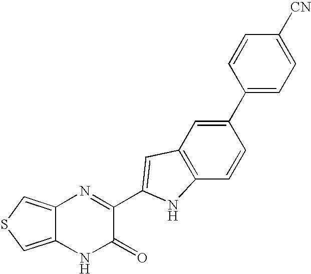 | | | 5, 1 (steps 2, 3), 2, 3 |
| 74 | 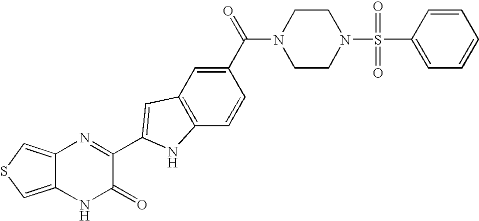 | | | 1, 2, 6, 3 |
| 75 | 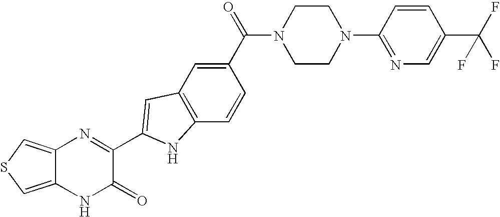 | | | 1, 2, 3 |
| 76 | 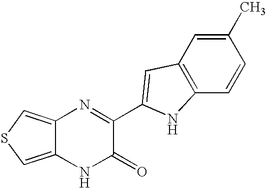 | | | 1 |
| 77 | 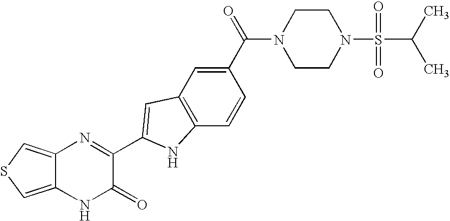 | | | 1, 2, 6, 3 |
| 78 | 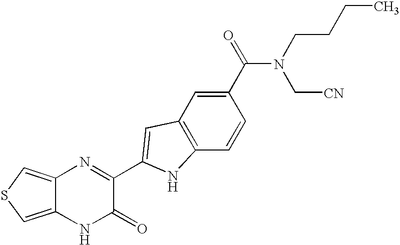 | | | 1, 2, 3 |
| 79 | 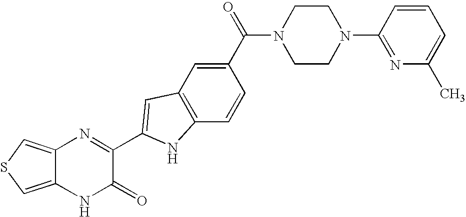 | | | 1, 2, 3 |
| 80 | 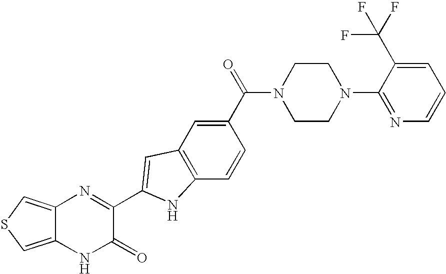 | | | 1, 2, 3 |
| 81 | 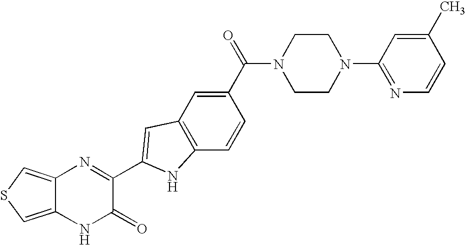 | | | 1, 2, 3 |
| 82 | 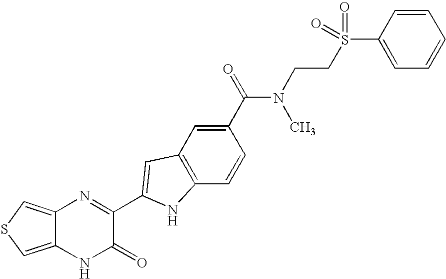 | | | 1, 2, 3 |
| 83 | 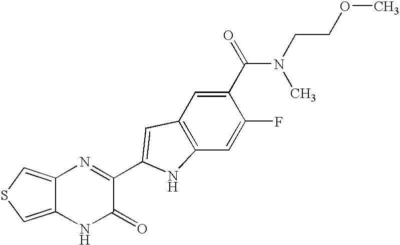 | | | 8 (steps 2, 3, 4), 1 (step 3), 2, 3 |
| 84 | 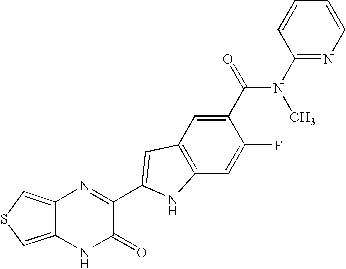 | | | 8 (steps 2, 3, 4), 1 (step 3), 2, 3 |
| 85 | 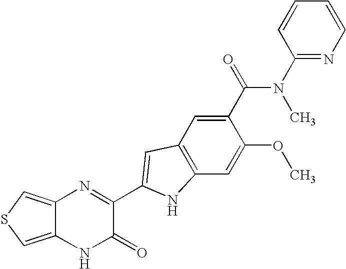 | | | 8 (steps 2, 3, 4), 1 (step 3), 2, 3 |
| 86 | 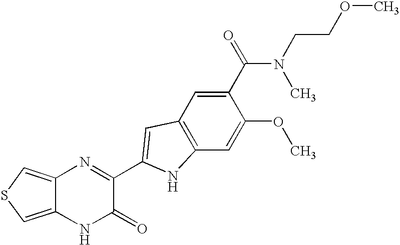 | | | 8 (steps 2, 3, 4), 1 (step 3), 2, 3 |
| 87 | 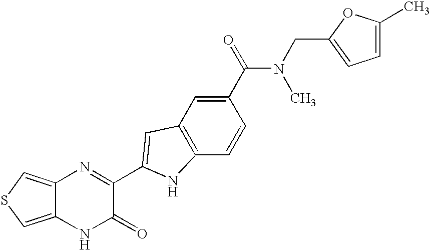 | | | 1, 2, 3 |
| 88 | 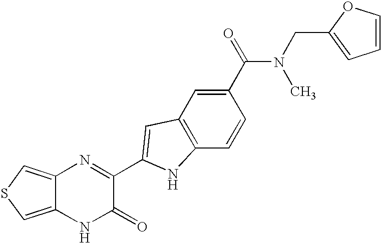 | | | 1, 2, 3 |
| 89 | 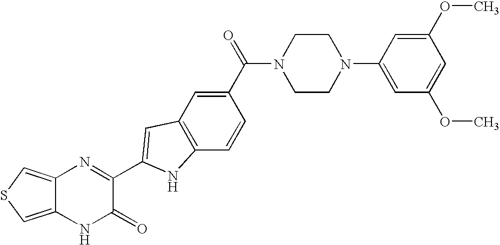 | | | 1, 2, 3 |
| 90 | 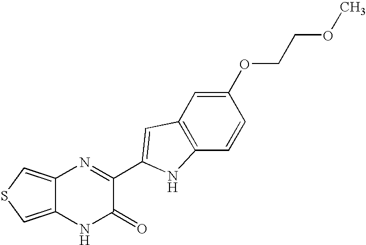 | | | 4, 1 (step 3) |
| 91 | 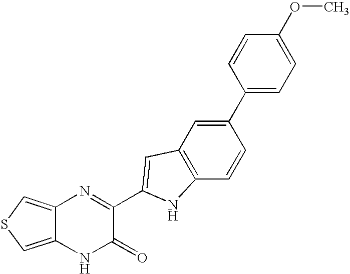 | | | 5, 1 (step 2, 3) |
| 92 | 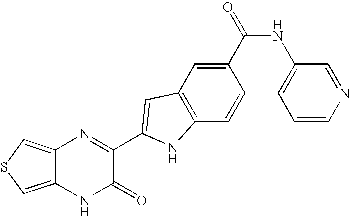 | | | 1, 2, 3 |
| 93 | 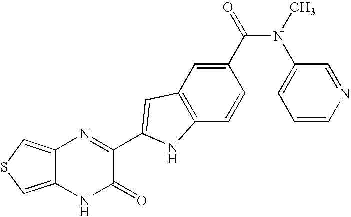 | | | 1, 2, 3 |
| 94 | 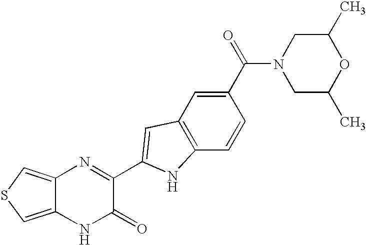 | | | 1, 2, 3 |
| 95 | 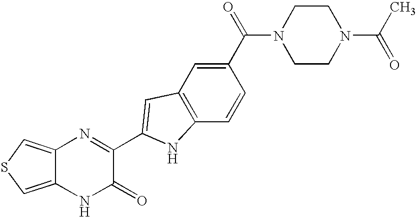 | | | 1, 2, 6, 3 |
| 96 | 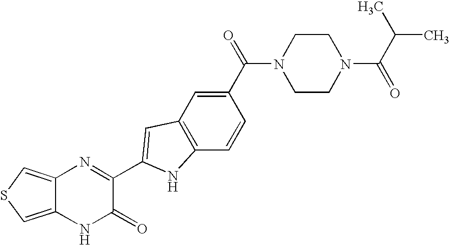 | | | 1, 2, 6, 3 |
| 97 | 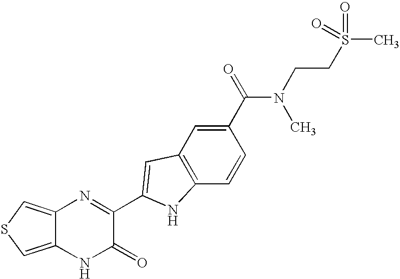 | | | 1, 2, 3 |
| 98 | 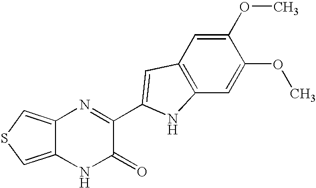 | | | 1 |
| 99 | 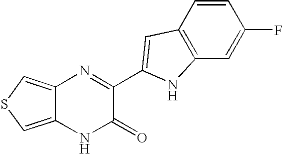 | | | 1 |
| 100 | 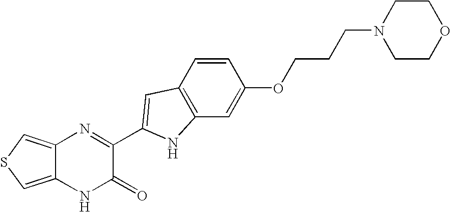 | | | 4, 1 (step 3) |
| 101 | 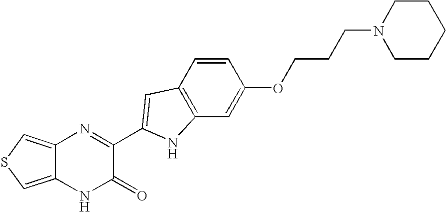 | | | 4, 1 (step 3) |
| 102 | 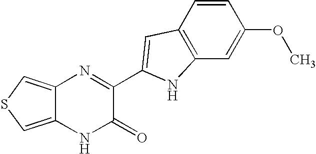 | | | 1 |
| 103 | 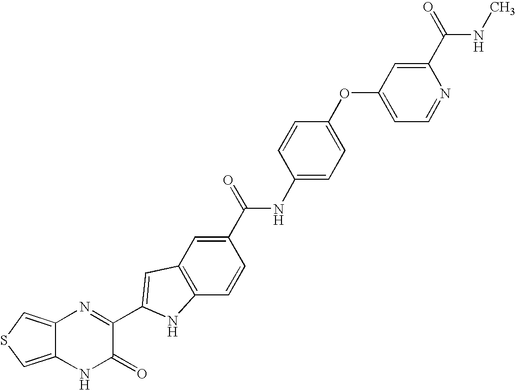 | | | 1, 2, 3 |
| 104 | 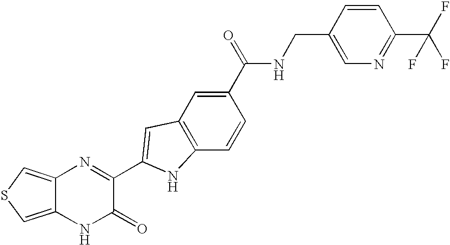 | | | 1, 2, 3 |
| 105 | 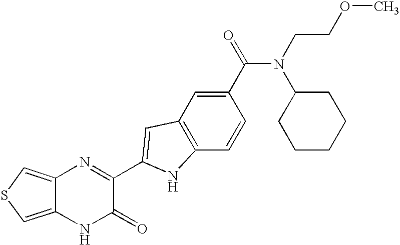 | | | 1, 2, 3 |
| 106 | 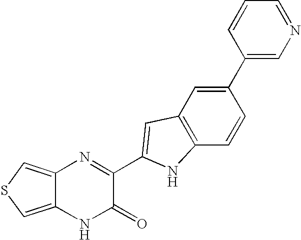 | | | 5, 1 (steps 2, 3) |
| 107 | 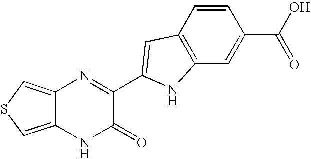 | | | 1, 2 |
| 108 | 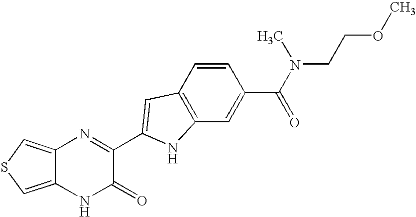 | | | 1, 2, 3 |
| 109 | 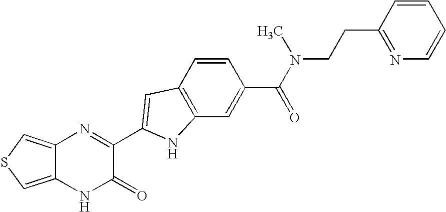 | | | 1, 2, 3 |
| 110 | 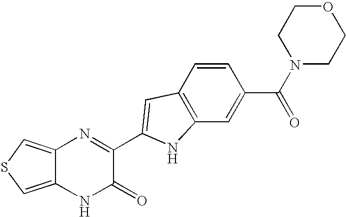 | | | 1, 2, 3 |
| 111 | 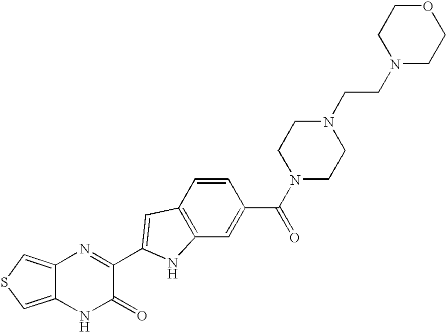 | | | 1, 2, 3 |
| 112 | 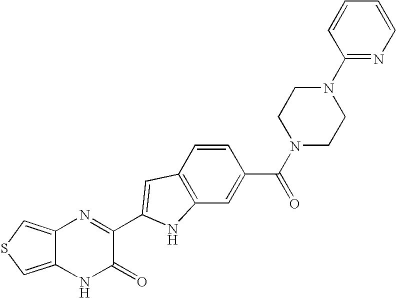 | | | 1, 2, 3 |
| 113 | 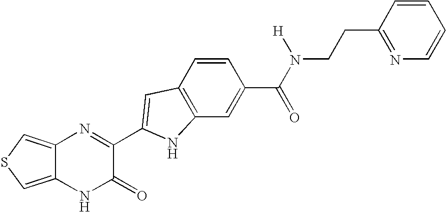 | | | 1, 2, 3 |
| 114 | 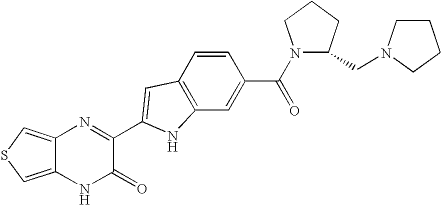 | | | 1, 2, 3 |
| 115 | 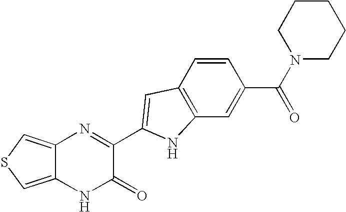 | | | 1, 2, 3 |
| 116 | 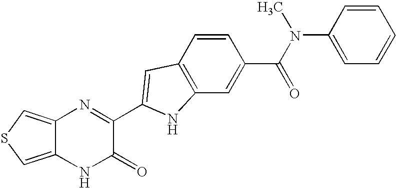 | | | 1, 2, 3 |
| 117 | 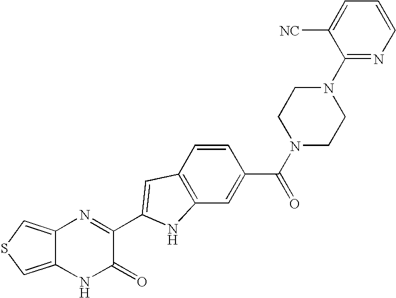 | | | 1, 2, 3 |
| 118 | 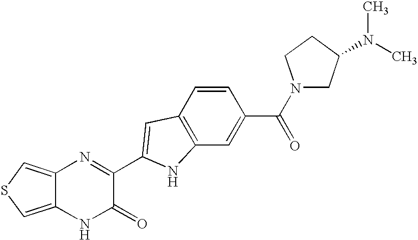 | | | 1, 2, 3 |
| 119 | 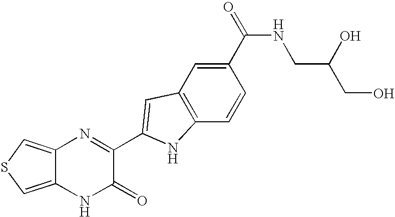 | | | 1, 2, 3 |
| 120 | 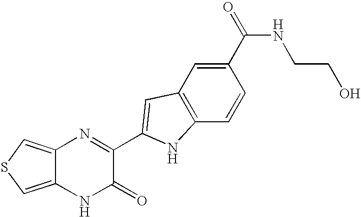 | | | 1, 2, 3 |
| 121 | 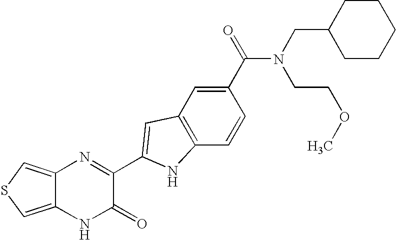 | | | 1, 2, 3 |
| 122 | 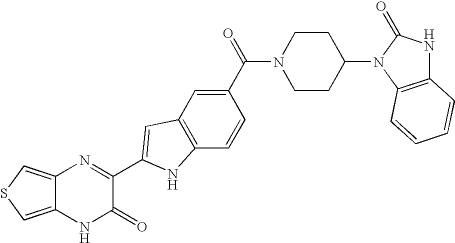 | | | 1, 2, 3 |
| 123 | 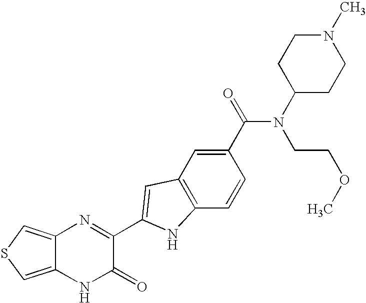 | | | 1, 2, 3 |
| 124 | 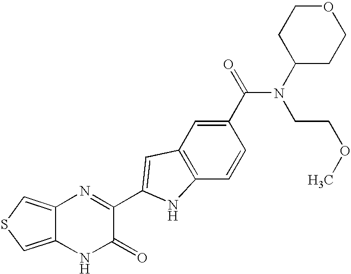 | | | 1, 2, 7, 3 |
| 125 | 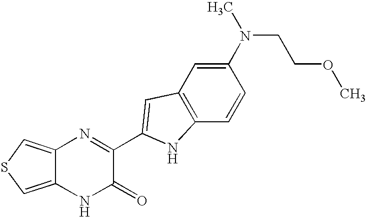 | | | 8, 1 (step 3) |
| 126 | 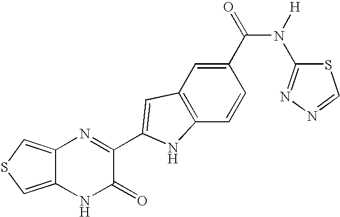 | | | 1, 2, 3 |
| 127 | 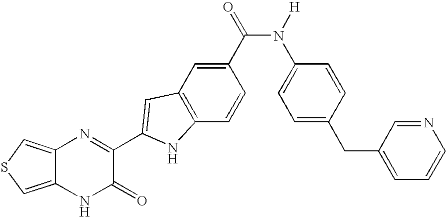 | | | 1, 2, 3 |
| 128 | 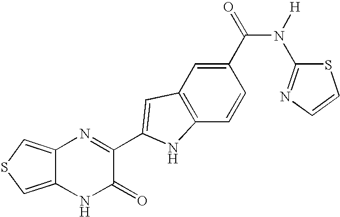 | | | 1, 2, 3 |
| 129 | 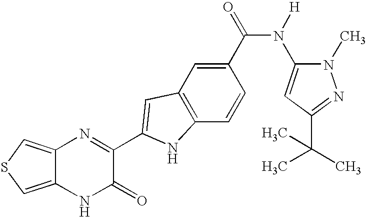 | | | 1, 2, 3 |
| 130 | 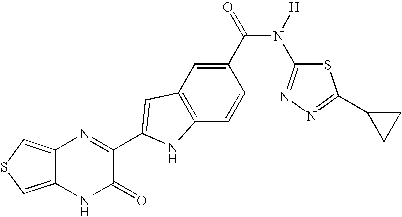 | | | 1, 2, 3 |
| 131 | 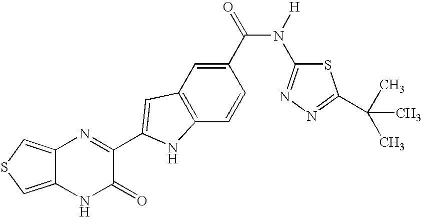 | | | 1, 2, 3 |
| 132 | 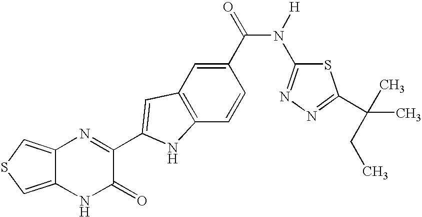 | | | 1, 2, 3 |
| 133 | 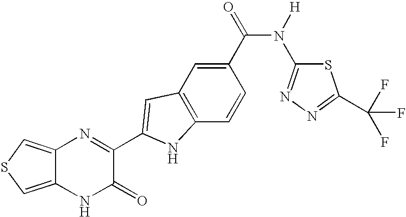 | | | 1, 2, 3 |
| 134 | 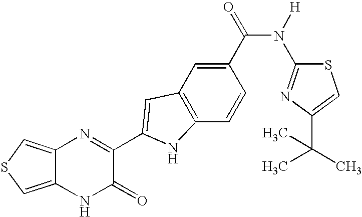 | | | 1, 2, 3 |
| 135 | 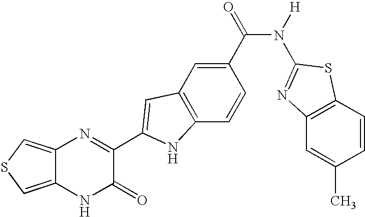 | | | 1, 2, 3 |
| 136 | 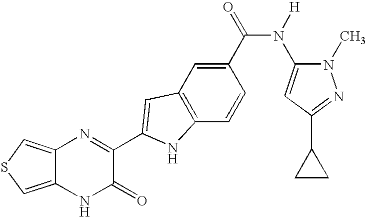 | | | 1, 2, 3 |
| 137 | 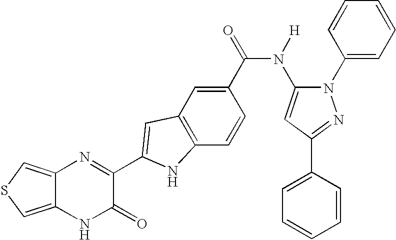 | | | 1, 2, 3 |
| 138 | 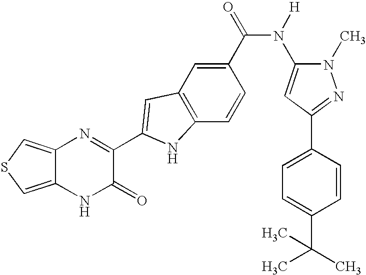 | | | 1, 2, 3 |
| 139 | 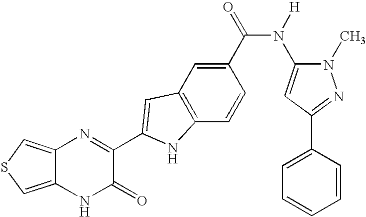 | | | 1, 2, 3 |
| 140 | 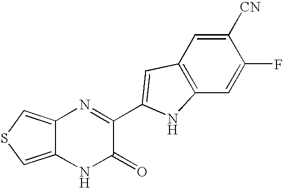 | | | 8, 1 (step 3) |
| 141 | 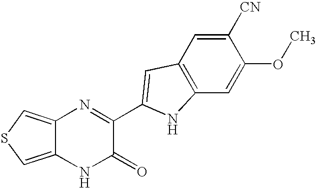 | | | 8, 1 (step 3) |
| 142 | 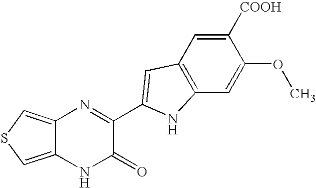 | | | 8, 1 (step 3), 2 |
| 143 | 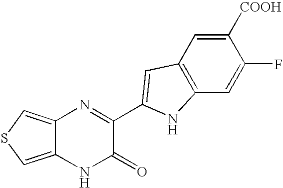 | | | 8, 1 (step 3), 2 |
| 144 | 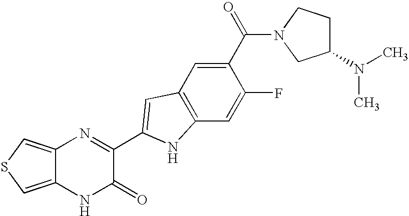 | | | 8, 1 (step 3), 2, 3 |
| 145 | 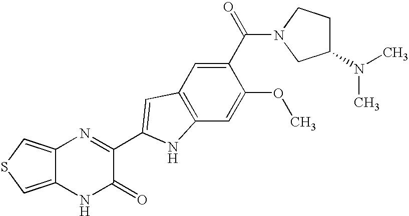 | | | 8, 1 (step 3), 2, 3 |
| 146 | 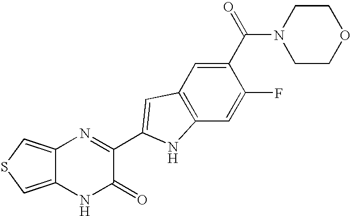 | | | 8, 1 (step 3), 2, 3 |
| 147 | 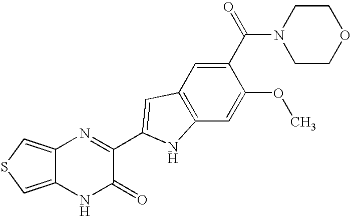 | | | 8, 1 (step 3), 2, 3 |
| 148 | 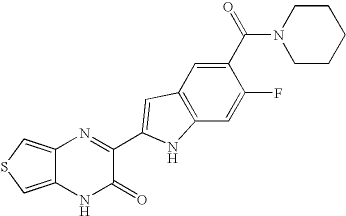 | | | 8, 1 (step 3), 2, 3 |
| 149 | 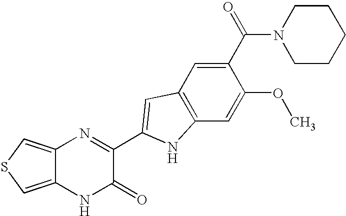 | | | 8, 1 (step 3), 2, 3 |
| 150 | 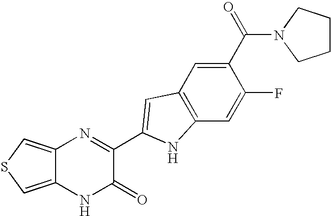 | | | 8, 1 (step 3), 2, 3 |
| 151 | 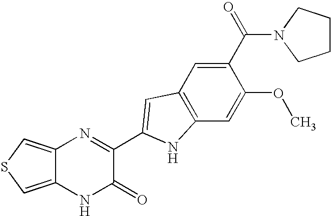 | | | 8, 1 (step 3), 2, 3 |
| 152 | 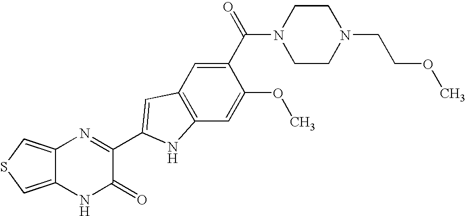 | | | 8, 1 (step 3), 2, 3 |
| 153 | 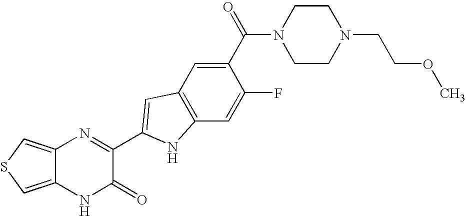 | | | 8, 1 (step 3), 2, 3 |
| 154 | 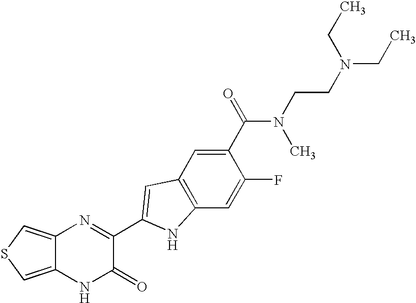 | | | 8, 1 (step 3), 2, 3 |
| 155 | 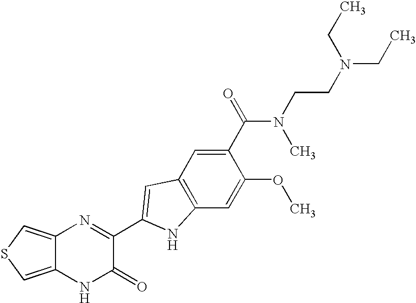 | | | 8, 1 (step 3), 2, 3 |
| 156 | 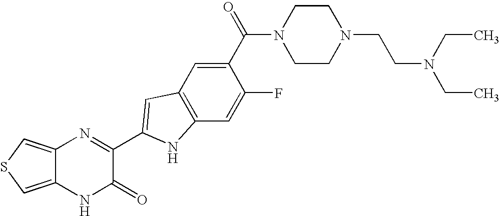 | | | 8, 1 (step 3), 2, 3 |
| 157 | 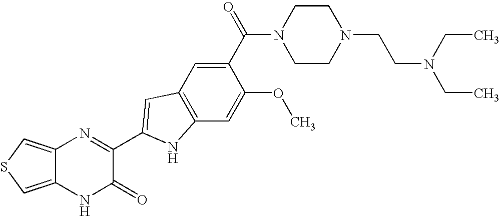 | | | 8, 1 (step 3), 2, 3 |
| 158 | 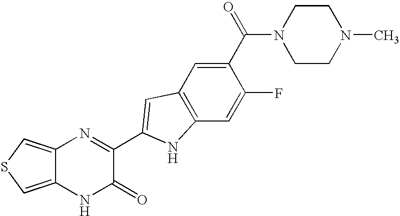 | | | 8, 1 (step 3), 2, 3 |
| 159 | 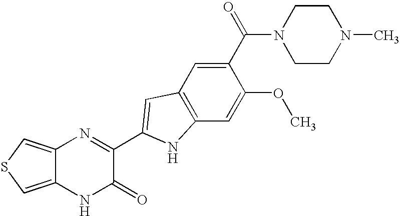 | | | 8, 1 (step 3), 2, 3 |
| 160 | 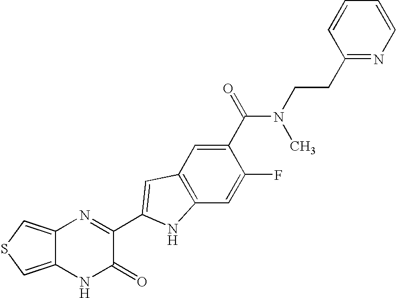 | | | 8, 1 (step 3), 2, 3 |
| 161 | 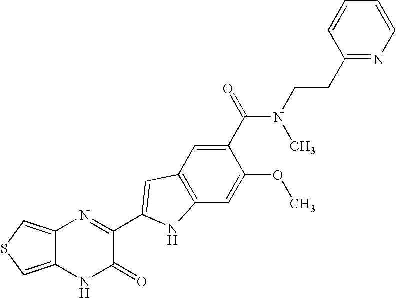 | | | 8, 1 (step 3), 2, 3 |
| 162 | 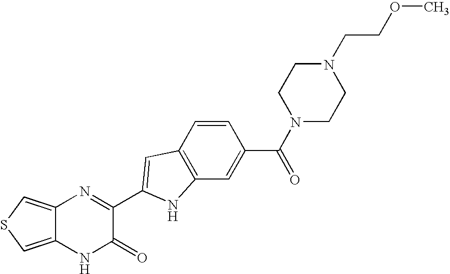 | | | 1, 2, 3 |
| 163 | 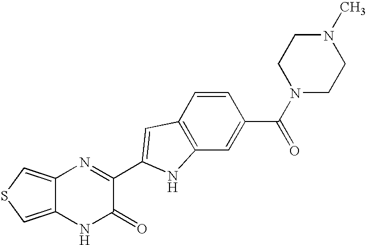 | | | 1, 2, 3 |
| 164 | 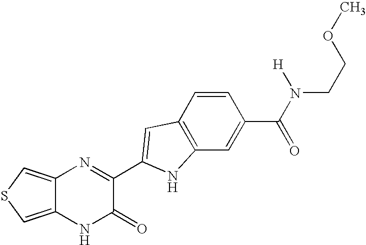 | | | 1, 2, 3 |
| 165 | 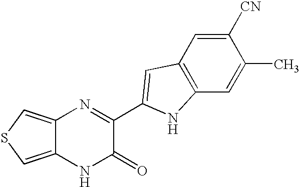 | | | 8, 1 (step 3) |
| 166 | 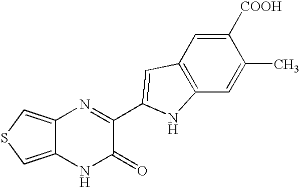 | | | 8, 1 (step 3), 2 |
| 167 | 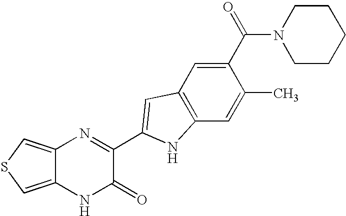 | | | 8, 1 (step 3), 2, 3 |
| 168 | 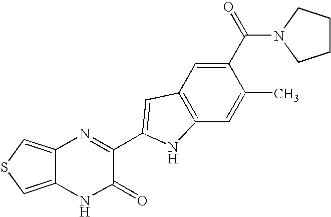 | | | 8, 1 (step 3), 2, 3 |
| 169 | 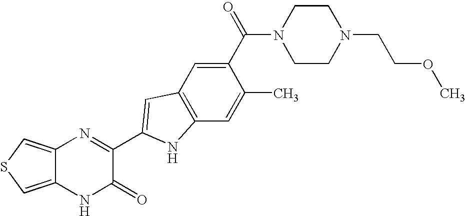 | | | 8, 1 (step 3), 2, 3 |
| 170 | 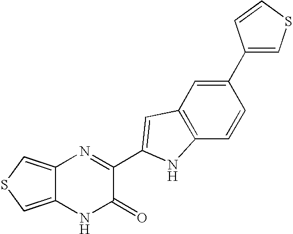 | | | 5, 1 (steps 2, 3) |
| 171 | 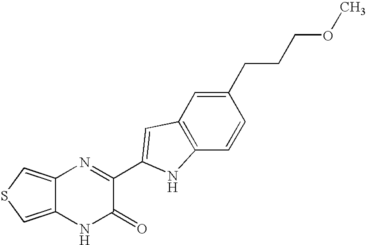 | | | 5, 1 (steps 2, 3) |
| 172 | 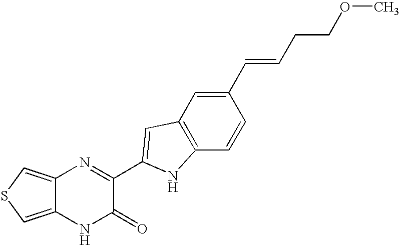 | | | 5, 1 (steps 2, 3) |
| 173 | 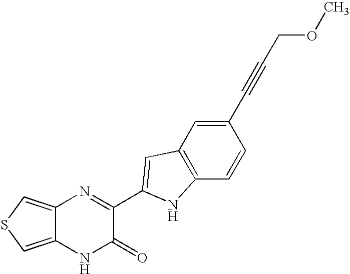 | | | 5, 1 (steps 2, 3) |
| 174 | 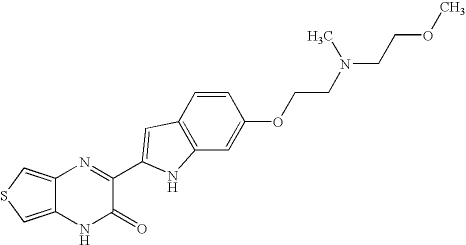 | | | 4, 1 (step 3) |
| 175 | 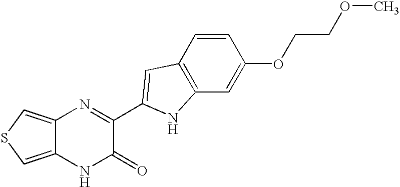 | | | 4, 1 (step 3) |
| 176 | 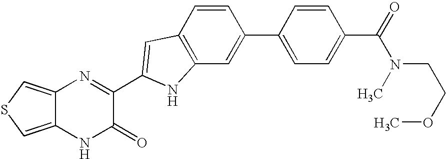 | | | 5, 1 (steps 2, 3), 2, 3 |
| 177 | 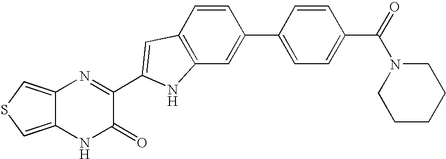 | | | 5, 1 (steps 2, 3), 2, 3 |
| 178 | 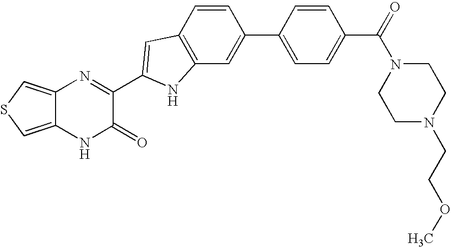 | | | 5, 1 (steps 2, 3), 2, 3 |
| 179 | 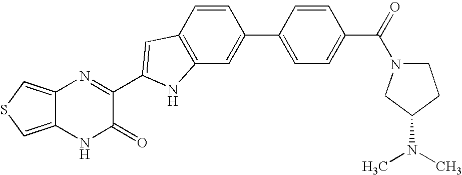 | | | 5, 1 (steps 2, 3), 2, 3 |
| 180 | 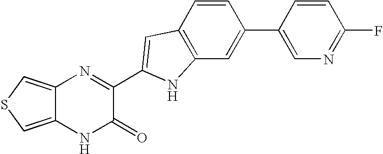 | | | 5, 1 (steps 2, 3) |
| 181 | 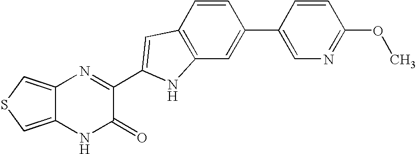 | | | 5, 1 (steps 2, 3) |
| 182 | 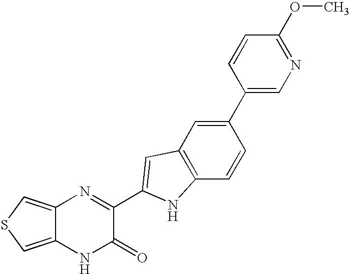 | | | 5, 1 (steps 2, 3) |
| 183 | 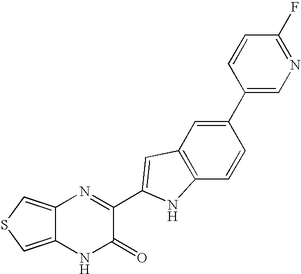 | | | 5, 1 (steps 2, 3) |
| 184 | 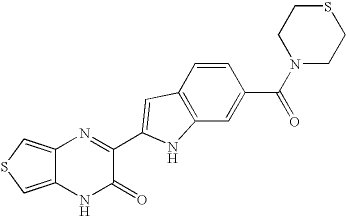 | | | 1, 2, 3 |
| 185 | 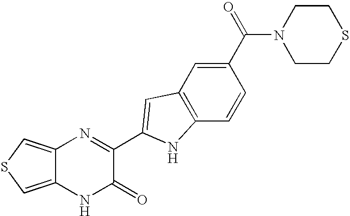 | | | 1, 2, 3 |
| 186 | 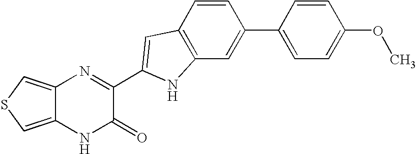 | | | 5, 1 (steps 2, 3) |
| 187 | 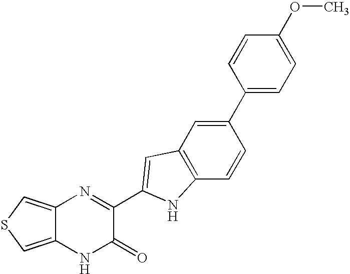 | | | 5, 1 (steps 2, 3) |

*Preparative methods: the numbers in this column indicate the order in which the processes analogous to the numbered specific examples (described below) would be followed, to make the specific compound identified in the row.

Asymmetry, i.e., where a compound's mirror image cannot be super-imposed on the compound, may be present in a compound of Formula (I) due to the inherent structure of the molecule. Examples of such asymmetric molecules include certain allenyl compounds. The compounds of this invention may also contain one or more asymmetric centers depending upon the location and nature of the various substituents selected. A molecule with a single asymmetric center may be a mixture of enantiomers (R,S), or may be a single (R) or (S) enantiomer. A molecule with more than one asymmetric center may be a mixture of diastereomers, or may be a single diastereomer. Additionally, a compound may exhibit asymmetry due to restricted rotation about a given bond, for example, the central bond adjoining two substituted aromatic rings of the specified compound. It is intended that all such configurations and conformations (including enantiomers, diastereomers, and other optical isomers) are included within the scope of the present invention. Separated, pure or partially purified stereo isomers of the compounds of Formula (I) are each included within the scope of the present invention. Preferred compounds are those with the absolute configuration or conformation which produces the more desirable biological activity.

The use of pharmaceutically acceptable salts of the compounds of this invention are also within the scope of this invention. The term “pharmaceutically acceptable salt” refers to either inorganic or organic salts of a compound of the present invention that have properties acceptable for the therapeutic use intended. For example, see S. M. Berge, et al. “Pharmaceutical Salts,” J. Pharm. Sci. 1977, 66, 1-19.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate. The term acid addition salts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and strearyl chlorides, bromides and iodides, aralkyl halides including benzyl and phenethyl bromides, and others.

The esters of appropriate compounds of this invention are pharmaceutically acceptable esters such as alkyl esters, including methyl, ethyl, propyl, isopropyl, butyl, isobutyl or pentyl esters, and the like. Additional esters such as phenyl-(C1-C5) alkyl may be used, although methyl ester is preferred.

Unless the context clearly indicates to the contrary, whenever the term “compounds of this invention,” “compounds of the present invention”, and the like, are used herein, they are intended to include the chemically feasible pharmaceutically acceptable salts and/or esters as well as all stereoisomeric forms of the referenced compounds.

Method of Making the Compounds of the Present Invention

In general, the compounds used in this invention may be prepared by standard techniques known in the art, by known processes analogous thereto, and/or by the processes described herein, using starting materials which are commercially available, producible according to routine, conventional chemical methods or the synthesis of which is described herein.

### Abbreviations and Acronyms

When the following abbreviations are used throughout the disclosure, they have the following meaning:
AcCl acetyl chloride
AcOH acetic acid
amu atomic mass unit
aq aqueous
Boc t-butoxycarbonyl
t-BuLi tert.-butyllithium
CDl carbonyl diimidazole
Celite® registered trademark of Celite Corp. brand of diatomaceous earth
conc concentrated
DMAP 4-(N,N-dimethyl)amino pyridine
DME dimethoxyethane
DMF N,N-dimethyl formamide
DMSO-d₆ dimethylsulfoxide-d₆
ESI electrospray ionization
Et₂O diethyl ethyl
EtOAc ethyl acetate
EtOH ethanol
h hour(s)
¹H NMR proton nuclear magnetic resonance
Hex hexanes
HPLC high performance liquid chromatography
LCMS liquid chromatography/mass spectroscopy
MeOH methanol
min minute(s)
MS mass spectrometry
Pd/C palladium on carbon
PyBOP Benzotriazol-1-yloxytris(pyrrolidino)phosphonium hexafluorophosphate
rb round-bottom
R_{f} TLC retention factor
rt room temperature
RT retention time (HPLC)
sat saturated
TBDMS tert-butyidimethylsilyl
TBDMSCI tert-butyldimethylsilyl chloride
TFA trifluoroacetic acid
THF tetrahydrofuran
TLC thin layer chromatography
TMS tetramethylsilane

Generally, compounds of the Formula (I) can be synthesized as shown in Scheme 1 or 2. Protection of a substituted indole of Formula (II) with a protecting group such as Boc produces an N-protected indole of Formula (III). The compound of Formula (III) can then be deprotonated and quenched with an electrophile, such as dimethyl oxalate to furnish a dicarbonyl indole compound of Formula (IV). The Formula (IV) compound can be condensed with 3,4-diaminothiophene dihydrobromide, available from Acros, Organics, Cat Number AC279470010, then deprotected if necessary, to generate a compound of Formula (I).

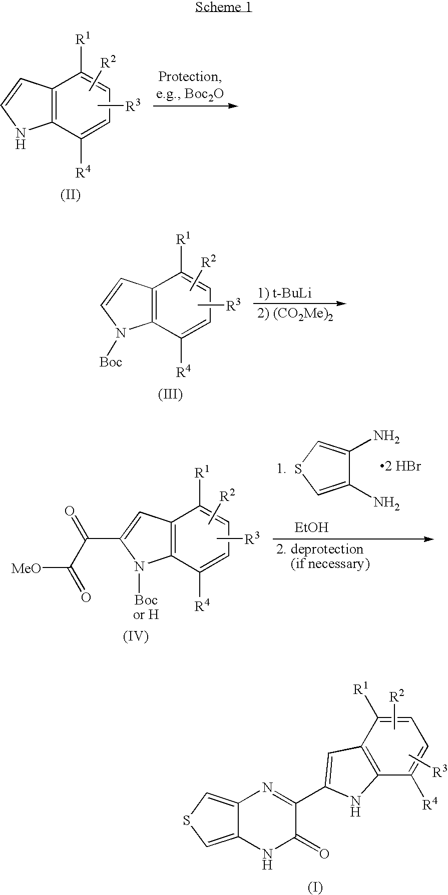

Compounds of Formula (II) and Formula (II) are either readily available, or can be prepared as shown in Schemes 3, 4, 7 or 8 below. Specifically, Scheme 3 illustrates synthesis of Formula (II) and (III) compounds where R² is optionally substituted phenyl or optionally substituted pyridyl. Scheme 4 illustrates synthesis of Formula (III) where R² is (C₁-C₆)alkoxy optionally substituted with 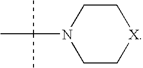
Scheme 7 illustrates synthesis of Formula (II) where R² is N[(C₁-C₃)alkyl]₂,), and Scheme 8 illustrates a general synthesis of Formula (II) compounds from readily available substituted anilines.

Scheme 2 shows how compounds of Formula (I), where R² is CN, can be converted to other compounds of Formula (I) where R² is C(O)R⁵ by standard functional group manipulation. For example, a cyanoindole (Ia) can be hydrolyzed under basic conditions such aqueous KOH to provide an indole carboxylic acid of Formula (Ib). Coupling of acid (Ib) with an amine, using a coupling agents such as PyBOP® provides a variety of amides of general Formula (Ic). 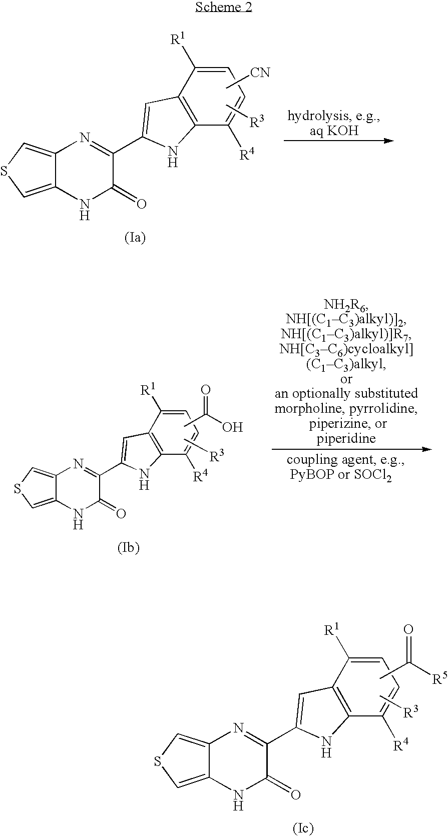
Preparation of Intermediates

Biaryl indole compounds of Formula (IIIb) where R² is an optionally substituted phenyl or pyridyl can be prepared as shown in Scheme 3. Performing a palladium catalyzed cross coupling between an indole boronic acid of Formula (IIb) and an optionally substituted phenyl or pyridyl bromide to provide the indole of Formula (IIc). Protection of (IIc) at the indole nitrogen provides the biaryl intermediate of Formula (IIIb).

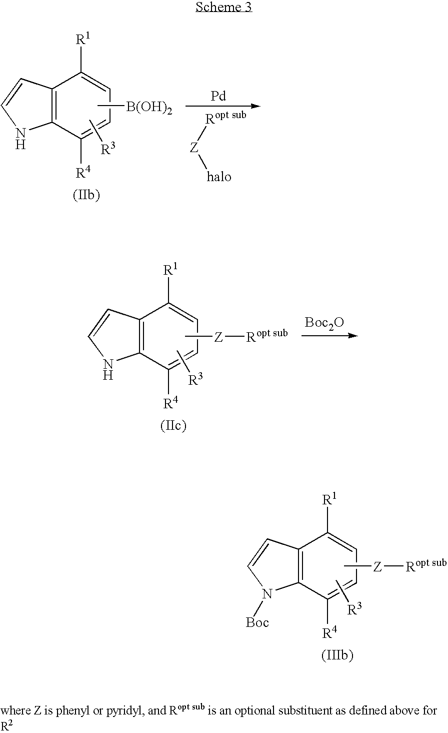

Intermediate indoles, used to prepare compounds of Formula (I), in which R² is an alkoxy group substituted by 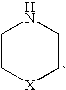
can be prepared from a hydroxyindole compound of Formula (IIIc) as shown in Scheme 4. Conversion of (IIIc) to an amine of Formula (IIIe) is accomplished by alkylation in two steps via an intermediate haloether (IIId). Step one is facilitated by the presence of a base such as Cs₂CO₃; step two is facilitated by a base such as triethyl amine. The Formula (IIIe) indole is carried on to final product of Formula (I) in the Schemes described above.

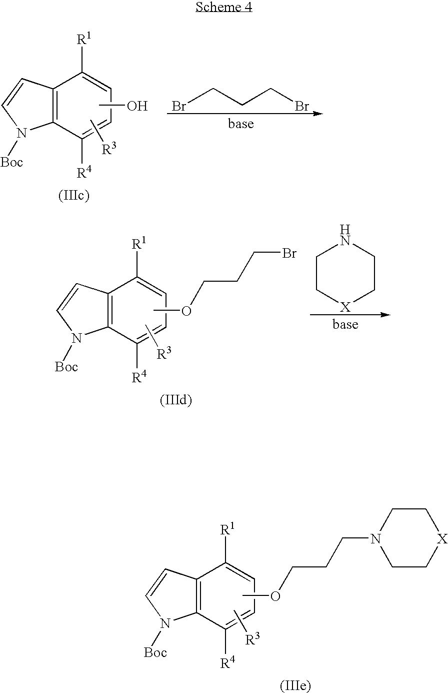

Substituted piperazines, used in the preparation of Formula (I) compounds in which R² is an alkyl or acyl group substituted by piperazine, can be prepared by conversion of a compound of Formula (V) to a sulfonamide (VI) upon treatment with methylsulfonyl chloride. The product, a N-Boc protected piperazine (VI) can be converted to a monosubstituted piperazine of Formula (VII) by subjecting (VI) to an acid such as TFA as shown in Scheme 5. The resulting Formula (VII) can be used, for example, in the last step in Scheme 2.

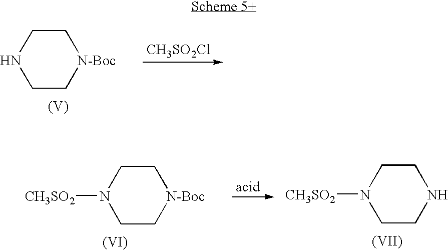

Amine derivatives of Formula (IX) can be prepared by conversion of a ketone of Formula (VIII) via reductive amination as shown in Scheme 6. This Scheme includes synthesis of the amine compounds that convert to the substituted piperidines of 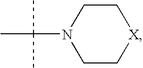
as well as to N[(C₃-C₆)cycloalkyl][(C₁-C₃)alkyl] and to substituted N[C₁-C₄)alkyl]₂, and can be inserted into, for example, the last step of Scheme 2.

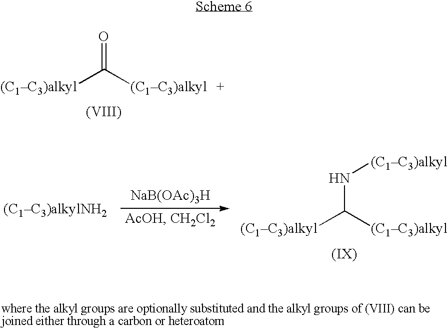

Compounds of Formula (IId) can be prepared as shown in Scheme 7. Conversion of a fluoronitrobenzene of Formula (X) to an aniline of Formula (XI) can be accomplished by displacement of the fluorine of (X). Nitroaniline (XI) can be converted to aminoindole (IId).

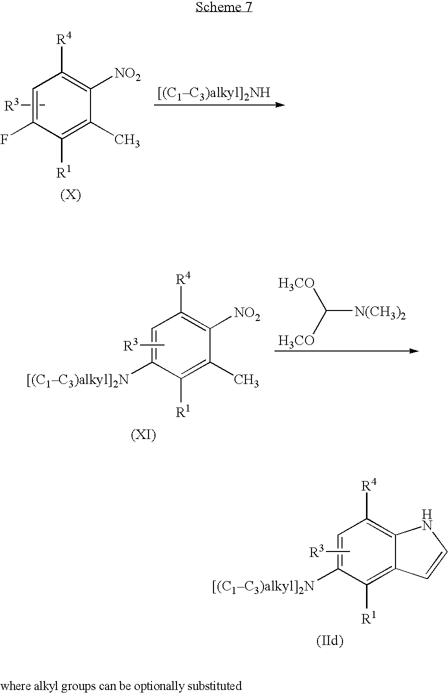

Compounds of Formula (IIe) can be synthesized from readily available substituted anilines of Formula (XII) as shown in Scheme 8. The anilines can be converted to diazonium salts of Formula (XIII), followed by reduction to substituted phenyl hydrazines of Formula (XIV). The hydrazines can be converted to phenyl hydrazones of Formula (XV) which can undergo an acid assisted cyclization to yield substituted indoles of Formula (IIe).

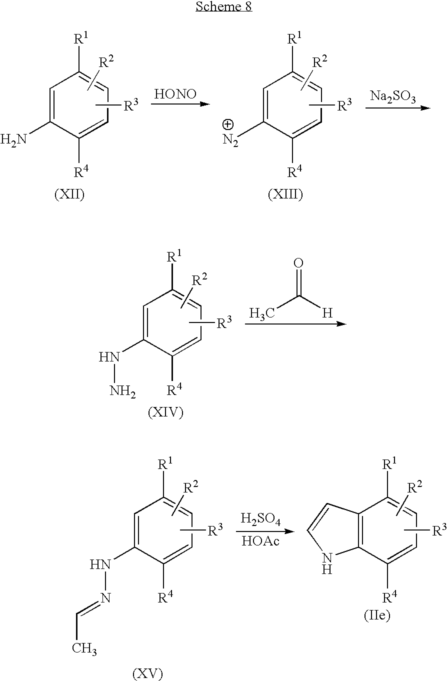

It is to be understood that sensitive or reactive substituents attached to intermediates or to compounds of Formula (I) may need to be protected and deprotected during the preparations described above. Protecting groups in general may be added and removed by conventional methods well known in the art [see, e.g., T. W. Greene and P. G. M. Wuts, Protective Groups in Organic Synthesis; Wiley: New York, (1999)].

In addition, it is to be understood that reaction conditions for N- or O-acylation, alkylation, or sulfonylation of the intermediates and of Formula (I) compounds using acyl halides, alkyl halides and sulfonyl halides, respectively, and a suitable base, are generally interchangeable, as is well known in the art. For example, conditions to effect N-acylation as described in any of the specific examples below can also be used to effect N-sulfonylation by substituting the appropriate sulfonyl halide for the acyl halide.

The following specific examples are presented to illustrate the invention described herein, but should not be construed as limiting the scope of the invention in any way.

The following specific examples are presented to illustrate the invention described herein, but should not be construed as limiting the scope of the invention in any way.

General Experimental Procedures

Electron impact mass spectra (EI-MS) were obtained with a Hewlett Packard 5989A mass spectrometer equipped with a Hewlett Packard 5890 Gas Chromatograph with a J & W DB-5 column (0.25 uM coating; 30 m×0.25 mm). The ion source was maintained at 250° C. and spectra were scanned from 50-800 amu at 2 sec per scan. High pressure liquid chromatography-electrospray mass spectra (LC-MS) were obtained using either a:

(A) Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.018% TFA. Gradient elution from 10% B to 95% over 3.5 minutes at a flowrate of 1.0 mL/min was used with an initial hold of 0.5 minutes and a final hold at 95% B of 0.5 minutes. Total run time was 6.5 minutes.

or

(B) Gilson HPLC system equipped with two Gilson 306 pumps, a Gilson 215 Autosampler, a Gilson diode array detector, a YMC Pro C-18 column (2×23 mm, 120 A), and a Micromass LCZ single quadrupole mass spectrometer with z-spray electrospray ionization. Spectra were scanned from 120-800 amu over 1.5 seconds. ELSD (Evaporative Light Scattering Detector) data was also acquired as an analog channel. The eluents were A: 2% acetonitrile in water with 0.02% TFA and B: 2% water in acetonitrile with 0.018% TFA. Gradient elution from 10% B to 90% over 3.5 minutes at a flowrate of 1.5 mL/min was used with an initial hold of 0.5 minutes and a final hold at 90% B of 0.5 minutes. Total run time was 4.8 minutes. An extra switching valve was used for column switching and regeneration.

Routine one-dimensional NMR spectroscopy was performed on 300 MHz Varian Mercury-plus spectrometers. The samples were dissolved in deuterated solvents obtained from Cambridge Isotope Labs, and transferred to 5 mm ID Wilmad NMR tubes. The spectra were acquired at 293 K. The chemical shifts were recorded on the ppm scale and were referenced to the appropriate solvent signals, such as 2.49 ppm for DMSO-d₆, 1.93 ppm for CD₃CN, 3.30 ppm for CD₃OD, 5.32 ppm for CD₂Cl₂ and 7.26 ppm for CDCl₃ for ¹H spectra, and 39.5 ppm for DMSO-d₆, 1.3 ppm for CD₃CN, 49.0 ppm for CD₃OD, 53.8 ppm for CD₂Cl₂ and 77.0 ppm for CDCl₃ for ¹³C spectra.

### EXAMPLE 1

### Preparation of 2-(3-oxo-3,4-dihydrothieno[3,4-b]pyrazin-2-yl)-1H-indole-5-carbonitrile

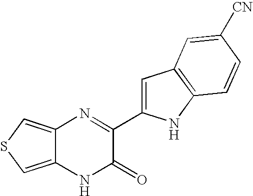

### Step 1. Preparation of tert-butyl 5-cyano-1H-indole-1-carboxylate

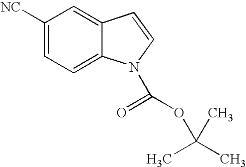

In a 100 mL round-bottom flask was placed 1H-indole-5-carbonitrile (2.0 g, 14.07 mmol) in 20 mL of anhydrous THF. To this solution was added DMAP (0.86 g, 7.03 mmol) and the mixture was allowed to stir for 0.5 h at rt. At this point, Boc₂O (3.07 g, 14.07 mmol) was added and the reaction stirred for an additional 2 h. The reaction was then quenched with Water and extracted twice with ethyl ether. The combined organic layers were washed successively with 1N HCl, water, and brine, then dried over MgSO₄ and concentrated to provide 3.26 g (96%) of the desired product as a white solid. ¹H-NMR (DMSO-d₆) δ 8.20-8.14 (m, 2H), 7.83 (d, 1H), 7.70 (d, 1H), 6.80 (d, 1H), 1.63 (s, 9H).

### Step 2. Preparation of methyl (5-cyano-1H-indol-2-yl)(oxo)acetate

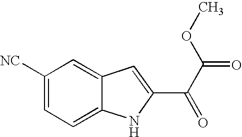

In a 100 mL round-bottom flask was placed 2.0 g (8.26 mmol, 1 equiv) of tert-butyl 5-cyano-1H-indole-1-carboxylate (step 1) 25 mL of THF. The mixture was cooled to −78° C. and 1.1 equiv (5.34 mL, 1.7 M in pentane) of t-BuLi was added dropwise. The mixture was allowed to stir for 1 h and 214 g (18.16 mmol, 2.2 equiv) of dimethyl oxalate in 5 mL of THF was added quickly in one portion. The reaction was then allowed to warm to 0° C. and stirred until complete, as monitored by TLC (about 2 h). The mixture was diluted with 30 mL of water and transferred to a separatory funnel where it was extracted with EtOAc (3×100 mL). The combined organic extract was washed with brine, dried (Na₂SO₄), filtered, and evaporated to give a brown residue. To the residue was added MeOH (10 mL) to give an insoluble yellow solid, which was filtered, washed with MeOH, dried, and purified to provide 0.44 g (24%) of the desired product as a yellow solid. ¹H-NMR (DMSO-d₆,) δ 12.63 (s, 1H), 8.40 (s, 1H), 7.77 (s, 1H), 7.65 (d, 1H), 7.60 (d, 1H), 3.93 (s, 3H).

### Step 3. Preparation of 2-(3-oxo-3,4-dihydrothieno[3,4-b]pyrazin-2-yl)-1H-indole-5-carbonitrile

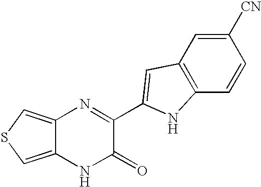

In a 250 mL round-bottom flask was placed 3.1 g (13.6 mmol, 1 equiv) of 5-cyano-2-[methoxy(oxo)acetyl]-1H-indole (step 2) and 3.75 g of 3,4-diaminothiophene dihydrobromide (13.6 mmol, 1 equiv.) in 75 mL of ethanol. The flask was equipped with a reflux condenser and heated at reflux for 2 h. The mixture was then allowed to cool to room temperature and precipitate was filtered and rinsed with an additional 20 mL of ethanol, dried in vacuo to provide 2.74 g (69%) of the desired product which was isolated as a black solid. ¹H-NMR (DMSO-d₆) δ 12.24 (s, 1H), 12.13 (s, 1H), 8.22 (s, 1H), 8.07 (s, 1H), 7.90 (s, 1H), 7.64-7.62 (d, 1H), 7.52-7.50 (d, 1H), 6.95 (s, 1H); LCMS RT=2.64 min; [M+H]⁺=293.2.

### EXAMPLE 2

### Preparation of 2-(3-oxo-3,4-dihydrothieno[3,4-b]pyrazin-2-yl)-1H-indole-5-carboxylic acid

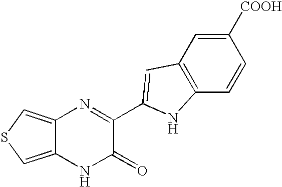

In a 250 mL round-bottom flask with condenser was placed 1.26 g of 2-(3-oxo-3,4-dihydrothieno[3,4-b]pyrazin-2-yl)-1H-indole-5-carbonitrile (Example 1) (4.22 mmol) in 100 mL of 4 M KOH. The mixture was heated at 120° C. for overnight. At this point, the reaction was allowed to cool to rt and acidified with conc HCl. The solids were filtered, rinsed with water and dried in vacuo at 60° C. to provide 1.3 g (100%) of the desired product which was isolated as a black solid. ¹H-NMR (DMSO-d₆) δ 12.24 (s, 1H), 11.94 (s, 1H), 8.32 (s, 1H), 8.03 (s, 1H), 7.90 (s, 1H), 7.80-7.76 (d, 1H), 7.55-7.50(d, 1H), 6.93 (s, 1H); LCMS RT=2.30 min; [M+H]⁺=312.0

### EXAMPLE 3

### Preparation of N-(2-methoxyethyl)-N-methyl-2-(3-oxo-3,4-dihydrothieno[3,4-b]pyrazin-2-yl)-1H-indole-5-carboxamide

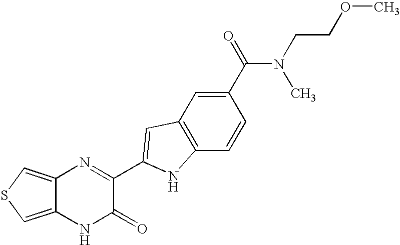

In a 25 mL round-bottom flask was placed 100 mg of 2-(3-oxo-3,4-dihydrothieno[3,4-b]pyrazin-2-yl)-1H-indole-5-carboxylic acid (Example 2) (0.32 mmol, 1 equiv.) and 0.07 mL of TEA (0.48 mmol, 1.5 equiv.) in 5 mL of DMF. To this was added 184 mg of PyBOP (0.35 mmol, 1.1 equiv.) and 0.04 ml of N-(2-methoxyethyl)methylamine (0.35 mmol). The reaction allowed to stir at rt overnight. The volatiles were removed and the residue was purified via preparative HPLC (CH₃CN/H₂O, 0.1% TFA). The desired fractions were combined and the CH₃CN removed in vacuo. The remaining aqueous solution was basified with saturated NaHCO₃. The precipitate was filtered, washed with water (5×) and dried in vacuo at 60° C. to provide 47 mg of a yellow solid (38%). ¹H-NMR (DMSO-d₆) δ 12.18 (s, 1H), 11.74 (s, 1H), 8.00 (s, 1H), 7.84 (s, 1H), 7.68 (s, 1H), 7.50-7.46 (d, 1H), 7.23-7.21 (d, 1H), 6.93 (s, 1H), 3.58-3.38 (m, 4H), 3.30-3.16 (broad, 3H), 2.99 (s, 3H); LCMS RT=2.31 min; [M+H]⁺=383.1.

### EXAMPLE 4

### Preparation of tert-butyl 2-[methoxy(oxo)acetyl]-5-[3-(4-morpholinyl)propoxy]-1H-indole-1-carboxylate

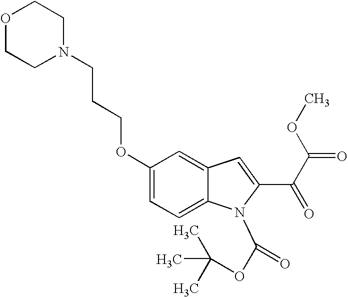

### Step 1. Preparation of tert-butyl 5-hydroxy-1H-indole-1-carboxylate

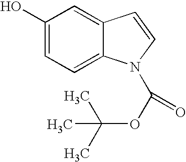

tert-Butyl 5-(benzyloxy)-1H-indole-1-carboxylate (5.75 g, 17.8 mmol), prepared according to the procedure described for Example 1, step 1, was added to a mixture of 10% Pd/C in EtOH. Ammonium formate was added and the reaction stirred for 6 h. The mixture was filtered through Celite® under a blanket of argon and the solvents were then removed. The residue was purified by flash chromatography to yield 3.5 g of tert-butyl 5-hydroxy-1H-indole-1-carboxylate (74%). ¹H-NMR (DMSO-d₆) δ 9.19 (s, 1H), 7.84-7.78 (d, 1H), 7.58-7.52 (d, 1H), 6.91 (s, 1H), 7.78-7.69 (m, 1H), 6.65-6.42 (m, 1H), 1.68-1.59 (s, 9H).

### Step 2. Preparation of tert-butyl 5-(3-bromopropoxy)-1H-indole-1-carboxylate

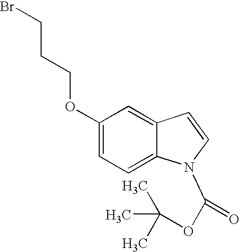

In a 250 mL flask was placed tert-butyl 5-(benzyloxy)-1H-indole-1-carboxylate (3.3 g, 14 mmol) in 100 mL of acetone. 1,3-Dibromopropane (5.74 mL, 56.6 mmol) was added, followed by cesium carbonate (5.5 g, 17 mmol). The reaction was heated to reflux for 5 h. The reaction was cooled to room temperature and diluted with water (200 mL). The mixture was transferred to a separatory funnel and extracted with ethyl acetate (2×150 mL). The combined organics were dried (MgSO₄), filtered, and evaporated. The residue was then purified via flash chromatography to provide 4.7 g of tert-butyl 5-(3-bromopropoxy)-1H-indole-1-carboxylate (94%). ¹H-NMR (DMSO-d₆) δ 7.99-7.89 (d, 1H), 7.61 (s, 1H), 7.17 (s, 1H), 6.98-6.91 (d, 1H), 6.62 (s, 1H), 4.16-4.05 (t, 2H), 3.64 (t, 2H), 2.37-2.20 (m, 2H). LCMS RT=3.55 min; [M]⁺=254.1.

### Step 3. Preparation of tert-butyl 5-[3-(4-morpholinyl)propoxy]-1H-indole-1-carboxylate

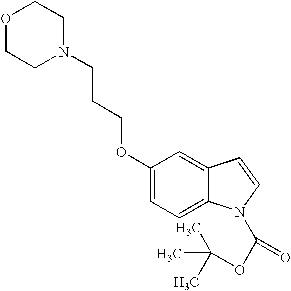

In a 250 mL flask was placed tert-butyl 5-(3-bromopropoxy)-1H-indole-1-carboxylate (1.5 g, 4.2 mmol) in 50 mL of THF. Morpholine (0.41 mL, 4.66 mmol) was added, followed by pyridine (0.38 mL, 4.66 mmol). The reaction was heated to reflux for 5 h. The reaction was cooled to rt and diluted with water (200 mL). The mixture was transferred to a separatory funnel and extracted with ethyl acetate (2×100 mL). The combined organics were dried (MgSO₄), filtered, and evaporated. The residue was then purified via flash chromatography to provide 1.1 g of tert-butyl 5-[3-(morpholinyl)propoxy)-1H-indole-1-carboxylate (72%). ¹H-NMR (DMSO-d₆) δ 7.93-7.85 (d, 1H), 7.59 (s, 1H), 7.09 (s, 1H), 6.93-6.85 (m, 1H), 6.59 (s, 1H), 4.06-3.97 (t, 2H), 3.57 (s, 4H), 2.46-2.23 (m, 6H), 1.92-1.83 (m, 2H), 1.62 (s, 9H). LCMS RT=0.61 min; [M+H]⁺=361.3.

### Step 4. Preparation of tert-butyl 2-[methoxy(oxo)acetyl]-5-[3-(4-morpholinyl)propoxy]-1H-indole-1-carboxylate

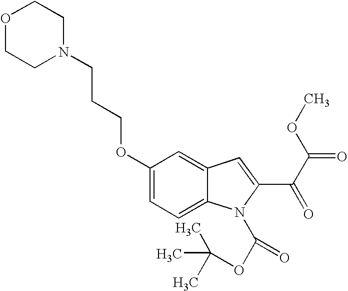

The compound was prepared by the method described for Example 1, step 2, using the product of Example 4, step 3 and dimethyl oxalate as starting materials. ¹H-NMR (DMSO-d₆) δ 7.86-7.80 (d, 1H), 7.38 (s, 1H), 7.20-7.29 (m, 1H), 7.18-7.10 (d, 1H), 4.06-3.99 (t, 2H), 3.80 (s, 3H), 3.57 (s, 4H), 2.47-2.24 (m, 6H), 1.96-1.83 (m, 2H), 1.59 (s, 9H).

### EXAMPLE 5

### Preparation of tert-butyl 5-(4-cyanophenyl)-1H-indole-1-carboxylate

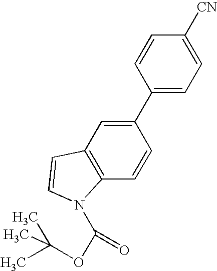

### Step 1. Preparation of 4-(1H-indol-5-yl)benzonitrile

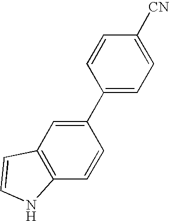

N₂ was bubbled through a solution of 5-indolylboronic acid (1.50 g, 9.32 mmol) in DME (55 mL) for 10 min. To this solution was added 1,1′-bis-(diphenylphosphine-ferrocene) dichloropalladium (II) complex with CH₂Cl₂ (1:1) (0.382 g, 0.440 mmol), 1.0 M solution of Na₂CO₃ (22 mL, 22 mmol) and 4-bromobenzonitrile (1.60 g, 8.87 mmol). N₂ was then bubbled through the reaction mixture for 10 min before the mixture was heated at 60° C. for 1 h. The reaction was quenched with H₂O and extracted with EtOAc (3×). The combined organic layers were washed with H₂O, brine, dried (MgSO₄), and concentrated to provide 2.24 g of crude brownish solid residue which was used in next step reaction without purification. ¹H-NMR (DMSO-d₆) δ 11.24 (s 1H), 7.91 (s, 1H), 7.85 (s, 4H), 7.47-7.45 (m, 2H), 7.39 (d, 1H), 6.49 (d, 1H).

### Step 2. Preparation of tert-butyl 5-(4-cyanophenyl)-1H-indole-1-carboxylate

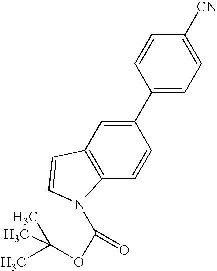

In a 100 mL rb flask was placed 4-(1H-indol-5-yl)benzonitrile (2.24 g, 10.3 mmol) in 100 mL of anhydrous THF. To this solution was added DMAP (0.630 g, 5.13 mmol) and the mixture allowed to stir for 0.5 h at rt. Boc₂O (2.24 g, 10.3 mmol) was added and the reaction stirred for 2 h. The reaction was then quenched with H₂O and extracted with Et₂O (2×). The combined organic layers were washed with 1N HCl, H₂O (2×), brine, dried (MgSO₄), and concentrated to provide 2.20 g (67%) of an off-white solid. ¹H-NMR (DMSO-d₆) δ 8.13-8.11 (d, 1H), 8.00 (s, 1H), 7.90 (s, 4H), 7.72-7.68 (m, 2H), 6.77 (d, 1H), 1.63 (s, 9H).

### EXAMPLE 6

### Preparation of 1-(methylsulfonyl)piperazine

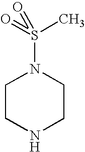

### Step 1. Preparation of tent-butyl 4-(methylsulfonyl)piperazine-1-carboxylate

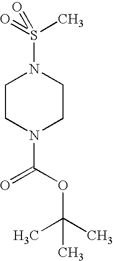

To a solution of tert-butyl piperazine-1-carboxylate (0.60 g, 3.2 mmol) in CH₂Cl₂ (10 mL) was added Et₃N (0.65 g, 6.4 mmol). The mixture was stirred for 10 min before methanesulfonyl chloride (0.40 g, 3.5 mmol) was added and the mixture allowed to stir overnight at rt. The reaction was quenched with H₂O and extracted with CH₂Cl₂ (2×). The combined organic layers were washed with H₂O, brine, dried (MgSO₄), filtered and concentrated to provide 0.80 g of an off-white solid (93%). ¹H-NMR (DMSO-d₆) δ 3.41-3.38 (t, 4H), 3.08-3.04 (t, 4H), 2.85 (s, 3H), 1.40 (s, 9H).

### Step 2. Preparation of 1-(methylsulfonyl)piperazine

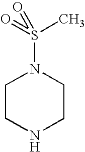

To a solution of tert-butyl 4-(methylsulfonyl)piperazine-1-carboxylate (0.80 g, 3.0 mmol) in CH₂Cl₂ (10 mL) was added TFA (1 mL). The mixture was stirred at rt for 3 h before the volatiles were removed. Et₂O was added to the residue then removed in vacuo to provide a yellow residue. Et₂O was added and the mixture was sonicated. The white solid precipitate was filtered, washed with Et₂O, and dried in an oven to provide 530 mg of an off-white solid (64%). ¹H-NMR (DMSO-d₆) δ 9.06 (s, 2H), 3.34-3.31 (m, 4H), 3.21-3.18 (m, 4H), 2.98 (s, 3H). LCMS [M+H]⁺=165.1.

### EXAMPLE 7

### Preparation of N-(2-methoxyethyl)cyclopentylamine

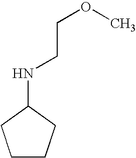

### Step 1. Preparation of N-(2-methoxyethyl)cyclopentylamine

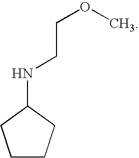

To a solution of cyclopentanone (2.00 g, 23.8 mmol) and 2-methoxyethylamine (1.78 g, 23.8 mmol) in CH₂Cl₂ (10 mL) was added sodium triacetoxyborohydride (7.05 g, 33.3 mmol) followed by AcOH (1.36 mL, 23.8 mmol). The reaction mixture was stirred at rt overnight. The reaction was quenched by adding sat NaHCO₃ and extracted with CH₂Cl₂ (2×). The combined organic layers were washed with brine, dried (MgSO₄), and concentrated to provide 0.70 g (21%) of crude free base as an yellowish oil which was used in next step reaction without further purification. ¹H-NMR (DMSO-d₆) δ 3.37-3.33 (m, 2H), 3.21 (s, 3H), 2.98-2.95 (m, 1H), 2.63-2.58 (t, 2H), 1.71-1.62 (m, 2H), 1.59-1.52 (m, 2H), 1.43-1.38 (m, 2H), 1.27-1.17 (m, 2H). LCMS RT=0.76 min; [M+H]⁺=144.2.

### EXAMPLE 8

### Preparation of tert-butyl 5-[(2-methoxyethyl)(methyl)amino]-2-[methoxy(oxo)acetyl]-1H-indole-1-carboxylate

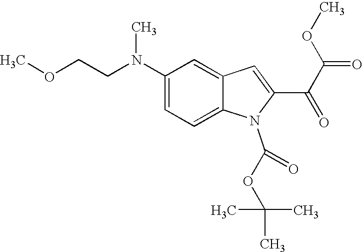

### Step 1. Preparation of N-(2-methoxyethyl)-N-3-dimethyl-4-nitroaniline

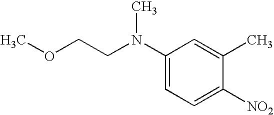

To a round bottom flask equipped with a reflux condenser was added 5-fluoro-2-nitrotoluene (10 g, 65.0 mmol) in 1-methyl-2-pyrrolidine (150 mL). N-(2-methoxyethyl)methylamine (21 mL, 200 mmol) was added to the stirring solution and the reaction was heated at 80° C. for 3 h. After cooling to rt, the product was purified by chromatography to yield 10.5 g (72%) of a yellow solid. ¹H NMR (DMSO-d₆): δ 2.62 (s, 3H), 3.01 (s, 3H), 3.24 (s, 3H), 3.40-3.45 (m, 2H), 3.57-3.61 (m, 2H), 6.58-6.62 (m, 2H).

### Step 2. N-(2-methoxyethyl)-N-methyl-1H-indol-5-amine

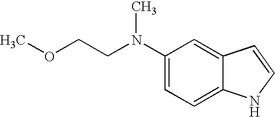

To a round bottom flask equipped with a reflux condenser was charged with N-(2-methoxyethyl)-N-3-dimethyl-4-nitroaniline (9.5 g, 42 mmol) and DMF (200 mL). N,N-dimethylformamide dimethylacetal (6.0 g. 50 mmol) and pyrrolidine (3.6 g, 50 mmol) were added and the reaction was heated at reflux for 3 h. After cooling to rt, the volatile components were removed in vacuo and the oily residue was dissolved in DMF (100 mL). The solution was added to 10% Pd/C (950 mg) under argon. The atmosphere was converted to H₂ with a balloon and the reaction allowed to stir at rt for 17 h. The H₂ was then removed and the mixture filtered through Celite® under a blanket of argon. The solvents were then removed and the product was purified by chromatography. The desired product was isolated as a red oil (7.9 g, 92%). ¹H-NMR (DMSO-d₆) δ 2.80-2.83 (m, 4H), 3.22 (s, 3H), 2.39-2.43 (m, 2H), 2.46-2.52 (m, 2H), 6.22-6.24 (m, 1H), 6.72-6.76 (d, 1H), 6.81 (s, 1H), 7.18-7.23 (m, 2H), 10.61 (br s, 1H); LCMS RT=0.27 min; [M+H]⁺=205.09.

### Step 3. Preparation of tert-butyl 5-[(2-methoxyethyl)(methyl)amino]-1H-indole-1-carboxylate

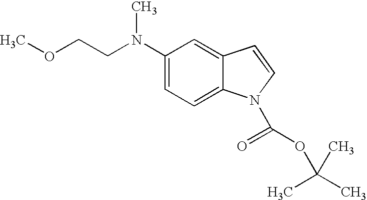

Using the method described in Example 1 Step 1, tert-butyl 5-[(2-methoxyethyl)(methyl)amino]-1H-indole-1-carboxylate was obtained as a colorless solid (7.2 g, 61%) from N-(2-methoxyethyl)-N-methyl-1H-indol-5-amine (7.8 g, 38 mmol). ¹H-NMR (DMSO-d₆) δ 1.63 (s, 9H), 2.90(s, 3H), 3.21 (s, 3H), 3.40-3.45 (m, 4H), 6.43-6.46 (m, 1H), 6.75-6.84 (m, 2H), 7.46-7.50 (d, 1H), 7.78-7.83 (d, 1H).

### Step 4. Preparation of tert-butyl 5-[(2-methoxyethyl)(methyl)amino]-2-[methoxy(oxo)acetyl]-1H-indole-1-carboxylate

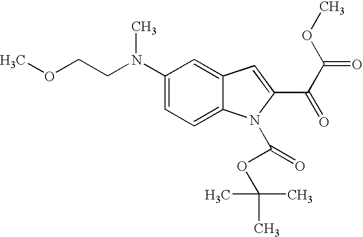

Using the method described in Example 1 Step 2, tert-butyl 5-[(2-methoxyethyl)(methyl)amino]-2-[methoxy(oxo)acetyl]-1H-indole-1-carboxylate was obtained as an oil (3.6 g, 80%) from tert-butyl 5-[(2-methoxyethyl)(methyl)amino]-1H-indole-1-carboxylate (3.5 g, 12 mmol). ¹H-NMR (DMSO-d₆) δ 1.59 (s, 9H), 3.93 (s, 3H), 3.21 (s, 3H), 3.41-3.58 (m, 4H), 6.85 (m, 1H), 7.05-7.13 (d, 1H), 7.25 (s, 1H), 7.75-7.79 (d, 1H).

Variations of the compounds of the invention can be readily prepared using the processes described above, or by other standard chemical processes known in the art, by employing appropriate starting materials that are readily available and/or are already described herein.

Generally, a desired salt of a compound of this invention can be prepared in situ during the final isolation and purification of a compound by means well known in the art. For example, a desired salt can be prepared by separately reacting the purified compound in its free base or free acid form with a suitable organic or inorganic acid, or suitable organic or inorganic base, respectively, and isolating the salt thus formed. In the case of basic compounds, for example, the free base is treated with anhydrous HCl in a suitable solvent such as THF, and the salt isolated as a hydrochloride salt. In the case of acidic compounds, the salts may be obtained, for example, by treatment of the free acid with anhydrous ammonia in a suitable solvent such as ether and subsequent isolation of the ammonium salt. These methods are conventional and would be readily apparent to one skilled in the art.

Esters of the compounds identified herein can be obtained by conventional means, for example, by reaction of a carboxylic acid compound with an alcohol facilitated by an acid catalyst, or by reaction of the carboxylic acid compound and alcohol under Mitsunobu conditions. These methods are conventional and would be readily apparent to one skilled in the art.

The purification of isomers of a compound of this invention, and the separation of said isomeric mixtures can be accomplished by standard techniques known in the art. Compositions of the compounds of this invention

The compounds of this invention can be utilized to achieve the desired pharmacological effect by administration to a patient in need thereof in an appropriately formulated pharmaceutical composition. A patient, for the purpose of this invention, is a mammal, including a human, in need of treatment (including prophylactic treatment) for the particular condition or disease. Therefore, the present invention includes pharmaceutical compositions that are comprised of a pharmaceutically acceptable carrier and a pharmaceutically effective amount of a compound, or salt or ester thereof, of the present invention. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, otically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients which can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl2F2, F2ClC—CClF2 and CClF3);

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate);

flavorants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures);

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose); tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch); tablet glidants (examples include but are not limited to colloidal silica, corn starch and talc);

tablet lubricants (examples include but are not limited to calcium stearate, magnesium stearate, mineral oil, stearic acid and zinc stearate); j tablet/capsule opaquants (examples include but are not limited to titanium dioxide); tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include be not limited to heptadecaethylene oxycetanol, lecithins, sorbitol monooleate, polyoxyethylene sorbitol monooleate, and polyoxyethylene stearate).

It is believed that one skilled in the art, utilizing the preceding information, can utilize the present invention to its fullest extent. Nevertheless, the following are examples of pharmaceutical formulations that can be used in the method of the present invention. They are for illustrative purposes only, and are not to be construed as limiting the invention in any way.

Pharmaceutical compositions according to the present invention can be illustrated as follows:

Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.

Lvophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.

Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate. Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Method of Treating Pharmacological Disorders

The present invention also relates to a method of using the compounds or compositions described herein for the treatment or prevention of, or in the manufacture of a medicament for treating or preventing, mammalian hyper-proliferative disorders. This method comprises administering to a patient (or a mammal) in need thereof, including a human, an amount of a compound, a pharmaceutically acceptable salt or ester thereof, or a composition of this invention which is effective to treat or prevent the disorder.

Hyper-proliferative disorders include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

The present invention also relates to a method for using the compounds of this invention as prophylactic or chemopreventive agents for prevention of the mammalian hyper-proliferative disorders described herein. This method comprises administering to a mammal in need thereof, including a human, an amount of a compound of this invention, or a pharmaceutically acceptable salt or ester thereof, which is effective to delay or diminish the onset of the disorder.

Examples of breast cancer include, but are not limited to invasive ductal carcinoma, invasive lobular carcinoma, ductal carcinoma in situ, and lobular carcinoma in situ.

Examples of hyper-proliferative disorders of the cardiovacular system include, but are not limited to, restenosis.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the nervous system include, but are not limited to glioblastoma.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal cancer, and lip and oral cavity cancer.

Lymphomas include, but are not limited to AIDS-related lymphoma, non-Hodgkin's lymphoma, cutaneous T-cell lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, and also exist with a similar etiology in other mammals which can also be treated by the administration of the compounds and/or pharmaceutical compositions of the present invention.

The utility of the compounds of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxol (Silvestrini et al. Stem Cells 1993, 11(6), 528-35), taxotere (Bissery et al. Anti Cancer Drugs 1995, 6(3), 339), and topoisomerase inhibitors (Edelman et al. Cancer Chemother. Pharmacol. 1996, 37(5), 385-93) were demonstrated with the use of in vitro tumor proliferation assays.

The present compounds and compositions exhibit anti-proliferative activity and are thus useful to treat the indications listed above, e.g. indications mediated by hyperproliferative disorders. Indications mediated by hyperproliferative disorders means diseases or conditions whose progression proceeds, at least in part, via proliferation.

The following assay is one of the methods by which compound activity relating to treatment of the disorders identified herein can be determined.

In Vitro Tumor Model Assay

Measurement of anti-proliferative activity can be evaluated as follows. A human tumor cell line such as HCT-116, was cultured under conditions recommended by the supplier (CCL-247, American Type Culture Collection, Manassas, Va., USA). To prepare the assay plates cells were removed from the culture dishes as a single cell suspension and plated at 5000 cell/well in a 96-well plate. Test compounds exemplified by Formula 1 above were dissolved in 100% dimethylsulfoxide at a concentration of 10 mmoles/L and diluted to the appropriated concentration such that the final dimethylsulfoxide concentration in the culture media did not exceed 0.25%. The day after cell plating, the test compounds were added to the culture medium at the appropriate dilutions, and the cells with the test compound were allowed to remain in contact under normal cell culture conditions for 72 hours. The inhibitory activity was measured using a CellTiter-Glo assay kit, using the instructions provided by the manufacture (Promega, Madison, Wis., USA). The % growth inhibition was calculated using the formula % inhibition=(value with test compound/value without test compound)×100.

Representative compounds of the invention were tested in the above assay and were found to be active.

Additionally, the compounds of this invention are useful in the prevention and/or treatment of, or in the manufacture of a medicament for treating, angiogenesis dependent disorders. A number of diseases are known to be associated with angiogenesis such as, for example, ocular neovascular disease, neovascular glaucoma, diabetic retinopathy, retrolental fibroplasia, hemangiomas, angiofibromas, psoriasis, age-related macula degeneration, haemangioblastoma, haemangioma, pain and inflammatory diseases such as rheumatoid or rheumatic inflammatory diseases including rheumatoid arthritis, as well as neoplastic diseases including, for example, so-called solid tumors and liquid tumors such as leukemias. As angiogenesis inhibitors, the compounds of this invention are also useful to control solid tumor growth such as breast, prostate, lung, pancreatic, renal, colon, and cervical cancer, melanoma, tumor metastasis, and the like as are well known in the art.

Tumors smaller than about 1-2 mm in diameter may receive oxygen and nutrients through diffusion directly into the tumor cells. However, angiogenesis is regarded as an absolute prerequisite for tumors that grow beyond that diameter. The principal mechanisms that play an important role in inhibition of tumor angiogenesis include inhibition of the growth of blood vessels, especially capillaries, into an avascular resting tumor, resulting in no net tumor growth due to the balance that is achieved between apoptosis and proliferation. Another route to treatment is through decreasing or preventing the migration of tumor cells throughout the body through the blood stream due to the inhibition of angiogenesis in relation to the tumor. Additionally, endothelial cell growth may be inhibited to avoid the paracrine growth-stimulating effect exerted on the surrounding tissue by the endothelial cells which normally line the blood vessels.

Measurement of anti-angiogenic activity can be evaluated as follows:

Xenograph Tumor Model Assay:

Female Ncr nude mice [Taconic Laboratories, NY] were inoculated subcutaneously with 5×106 MDA-MB-231 breast tumor cells (NCI, MD) on day 0. When tumors reached the size about 75 to 150 mm3, tumor-bearing animals were randomly divided into several groups with 10 mice per group and received the treatment with either vehicle or test compounds. All test compounds were formulated in PEG 400:Ethanol:50 mM methanesulfonic acid (40:10:50, v/v/v) vehicle, and given orally for 14 days. The dosing volumes were 0.1 mL-test article/10 g body weight or 10 mL/kg. During the course of the study, the length and width of each tumor was measured with electronic calipers every 2 or 3 days, and tumor size was calculated at each measuring time-point based on the formula of [length (mm)×width (mm)2]/2. Animal body weights were also recorded at the same time. All animals were observed for clinical signs daily after compound administration. At the end of the treatment period, tumors from both control animals and from animals treated with test compounds were resected and fixed in 10% buffered formalin and imbedded in paraffin. Tissue sections were prepared for immunohistochemistry and stained with anit-CD31 antibodies (sc-1506, Santa Cruz, Calif.) and developed using an ABC kit (Vector, Burlingame, Calif.) according to the manufacturer's instructions. The amount of CD31 staining as a percentage of the total area relative to untreated tumors was determined from images of sections using ImagePro Plus (Media Cybernetics, Silver Spring, Md.) software.

Representative compounds of the invention were tested in the above assay and were found be active in reducing tumor size and in inhibiting angiogenisis.

Based upon the above and other standard laboratory techniques known to evaluate compounds useful for the prevention and/or treatment of the diseases or disorders described above by standard toxicity tests and by standard pharmacological assays for the determination of the prevention and/or treatment of the conditions identified above in mammals, and by comparison of these results with the results of known medicaments that are used to treat these conditions, the effective dosage of the compounds of this invention can readily be determined for prevention and/or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 300 mg/kg, and preferably from about 0.10 mg/kg to about 150 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound of the present invention or a pharmaceutically acceptable salt or ester or composition thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with other anti-hyper-proliferative or other indication agents, and the like, as well as with admixtures and combinations thereof.

For example, optional anti-hyper-proliferative agents which can be added to the composition include but are not limited to compounds listed on the cancer chemotherapy drug regimens in the 11th Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the is invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of this invention include but are not limited to other anti-cancer agents such as epothilone, irinotecan, raloxifen and topotecan.

It is believed that one skilled in the art, using the preceding information and information available in the art, can utilize the present invention to its fullest extent.

It should be apparent to one of ordinary skill in the art that changes and modifications can be made to this invention without departing from the spirit or scope of the invention as it is set forth herein.

What is claimed is:

1. A compound of Formula I 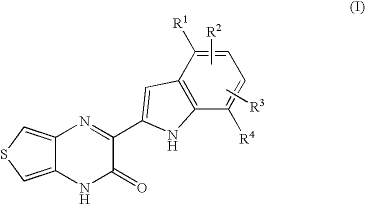
wherein
R¹ is selected from H, F, and Cl;
R² is selected from H, OH, CN, halo, C(O)R⁵, thienyl, pyrimidinyl, oxazolyl, furanyl,
(C₁-C₃)alkyl, (C₂-C₆)alkenyl and (C₂-C₆)alkynyl, each optionally substituted with up to two substituents selected from OH, halo, and
(C₁-C₃)alkoxy optionally substituted with (C₁-C₃)alkoxy,
(C₁-C₆)alkoxy optionally substituted with (C₁-C₃)alkyl, (C₁-C₃)alkoxy, pyrrolidinyl, 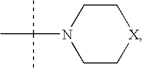
and N[(C₁-C₃)alkyl]₂ where each alkyl group is independently optionally substituted with a substituent selected from (C₁-C₃)alkyl, (C₁-C₃)alkoxy OH, halo, 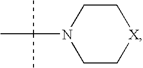
and phenyl,
N[(C₁-C₄)alkyl]₂ where each alkyl group is independently optionally substituted with up to two substituents independently selected from OH, (C₁-C₃)alkyl, halo, (C₁-C₃)alkoxy, and phenyl,
pyridyl optionally substituted with up to two substituents independently selected from (C₁-C₃)alkyl, (C₁-C₃)alkoxy, and halo,
phenyl optionally substituted with up to two substituents independently selected from (C₁-C₃)alkoxy, CN, halo, 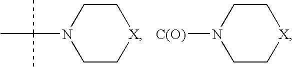
C(O)N[(C₁-C₃)alkyl]₂ where each alkyl is optionally substituted with (C₁-C₃)alkoxy, and
pyrrolidinyl optionally substituted with N[(C₁-C₃)alkyl]₂;
R³ is selected from H, halo, (C₁-C₃)alkyl, and (C₁-C₃)alkoxy;
R⁴ is selected from H, F, and Cl;
R⁵ is selected from OH, NHR⁶,
N[(C₁-C₃)alkyl]R⁷ where said alkyl is optionally substituted with up to one substituent selected from (C₁-C₃)alkyl and (C₁-C₃)alkoxy,
N[(C₁-C₃)alkyl]₂ where each alkyl is optionally substituted with up to two substituents independently selected from CN, OH, (C₁-C₃)alkoxy, N[(C₁-C₃)alkyl]₂, pyridyl, phenyl, S(O)₂(C₁-C₃)alkyl, tetrahydrofuryl, S(O)₂-phenyl, (C₃-C₆)cycloalkyl, and
furyl optionally substituted with (C₁-C₃)alkyl,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl, 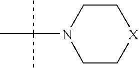
optionally substituted with up to two substituents independently selected from N[(C₁-C₃)alkyl]₂, C(O)(C₁-C₃)alkyl, pyrrolidinyl, S(O)₂(C₁-C₃)alkyl, S(O)₂-phenyl, 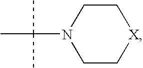
oxo-dihydrobenzimidazolyl, pyrazinyl, C(O)NH₂, C(O)NH-phenyl, C(O)-furanyl, C(O)NH(C₁-C₃)alkyl,
(C₁-C₃)alkyl optionally substituted with up to two substituents independently selected from OH, halo, (C₁-C₃)alkoxy, 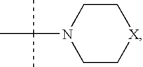
pyrrolidinyl, C(O)-pyrrolidinyl, 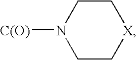
and N[(C₁-C₃)alkyl]₂,
phenyl optionally substituted with up to two substituents independently selected from (C₁-C₃)alkyl, (C₁-C₃)alkoxy, halo, CF₃, and CN, and
pyridyl optionally substituted with (C₁-C₃)alkyl, CF₃, and CN, and
pyrrolidinyl optionally substituted with up to two substituents independently selected from N[(C₁-C₄)alkyl]₂, C(O)NH₂, pyridyl, and
(C₁-C₃)alkyl optionally substituted with up to two substituents independently selected from (C₁-C₃)alkoxy, and pyrrolidinyl;
R⁶ is selected from H,
(C₁-C₄)alkyl optionally substituted with up to two substituents independently selected from OH, halo, (C₁-C₄)alkoxy, NHC(O)(C₁-C₃)alkyl, S—(C₁-C₃)alkyl, benzimidazolyl, thienyl, 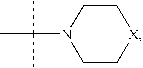
N[(C₁-C₄)alkyl)₂ ] where each alkyl is independently optionally substituted with up to two substituents independently selected from OH, (C₁-C₃)alkoxy, halo, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
indolyl optionally substituted up to two times with (C₁-C₃)alkyl,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, and halo,
indolyl optionally substituted up to two times with (C₁-C₃)alkyl,
pyrazolyl optionally substituted with up to two substituents independently selected from (C₁-C₄)alkyl, (C₃-C₆)cycloalkyl, and
phenyl optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl optionally substituted up to two times with (C₁-C₄)alkyl,
thiadiazolyl optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 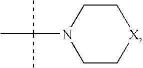
(C₁-C₄)alkoxy, O-pyridyl optionally substituted with C(O)NH(C₁-C₄)alkyl,
(C₁-C₄)alkyl optionally substituted with up to two substituents independently selected from pyridyl, OH, halo, and phenyl, and 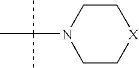
optionally substituted with up to two substituents independently selected from (C₁-C₃)alkyl, and (C₁-C₄)alkoxy,
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R⁷ is selected from (C₁-C₃)alkoxy, pyrrolidinyl, tetrahydropyranyl,
pyridyl optionally substituted with up to two substituents independently selected from (C₁-C₄)alkyl and (C₁-C₃)alkoxy,
pyranyl optionally substituted with up to two substituents independently selected from (C₁-C₄)alkyl and (C₁-C₃)alkoxy,
piperidinyl optionally substituted with up to two substituents independently selected from (C₁-C₃)alkyl, and (C₁-C₃)alkoxy, and
phenyl optionally substituted with up to two substituents independently selected from (C₁-C₃)alkoxy, and (C₁-C₃)alkyl; and
X is selected from O, S, CH₂ and NH;
with the proviso that when R¹ is F or Cl, then R⁴ must be H, and when R⁴ is F or Cl, then R¹ must be H;
or a pharmaceutically acceptable salt thereof.

2. A method of treating a disorder selected from a hyper-proliferative disorder and a disorder associated with angiogenesis, in a mammal in need thereof, comprising administering to said mammal an effective amount of a compound of Formula I 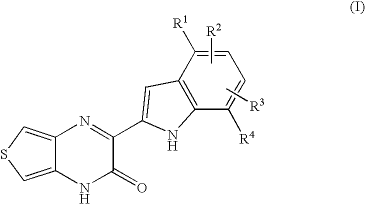
wherein
R¹ is selected from H, F, and Cl;
R² is selected from H, OH, CN, halo, C(O)R⁵, thienyl, pyrimidinyl, oxazolyl, furanyl,
(C₁-C₃)alkyl, (C₂-C₆)alkenyl and (C₂-C₆)alkynyl, each optionally substituted with up to two substituents selected from OH, halo, and
(C₁-C₃)alkoxy optionally substituted with (C₁-C₃)alkoxy,
(C₁-C₆)alkoxy optionally substituted with (C₁-C₃)alkyl, (C₁-C₃)alkoxy, pyrrolidinyl, 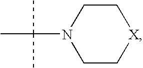
and N[(C₁-C₃)alkyl]₂ where each alkyl group is independently optionally substituted with a substituent selected from (C₁-C₃)alkyl, (C₁-C₃)alkoxy OH, halo, 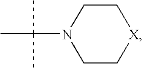
and phenyl,
N[(C₁-C₄)alkyl]₂ where each alkyl group is independently optionally substituted with up to two substituents independently selected from OH, (C₁-C₃)alkyl, halo, (C₁-C₃)alkoxy, and phenyl,
pyridyl optionally substituted with up to two substituents independently selected from (C₁-C₃)alkyl, (C₁-C₃)alkoxy, and halo,
phenyl optionally substituted with up to two substituents independently selected from (C₁-C₃)alkoxy, CN, halo, 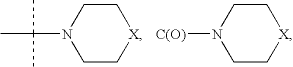
C(O)N[(C₁-C₃)alkyl]₂ where each alkyl is optionally substituted with (C₁-C₃)alkoxy, and
pyrrolidinyl optionally substituted with N[(C₁-C₃)alkyl]₂;
R³ is selected from H, halo, (C₁-C₃)alkyl, and (C₁-C₃)alkoxy;
R⁴ is selected from H, F, and Cl;
R⁵ is selected from OH, NHR⁶,
N[(C₁-C₃)alkyl]R⁷ where said alkyl is optionally substituted with up to one substituent selected from (C₁-C₃)alkyl and (C₁-C₃)alkoxy,
N[(C₁-C₃)alkyl]₂ where each alkyl is optionally substituted with up to two substituents independently selected from CN, OH, (C₁-C₃)alkoxy, N[(C₁-C₃)alkyl]₂, pyridyl, phenyl, S(O)₂(C₁-C₃)alkyl, tetrahydrofuryl, S(O)₂-phenyl, (C₃-C₆)cycloalkyl, and furyl optionally substituted with (C₁-C₃)alkyl,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl, 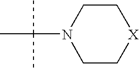
optionally substituted with up to two substituents independently selected from N[(C₁-C₃)alkyl]₂, C(O)(C₁-C₃)alkyl, pyrrolidinyl, S(O)₂(C₁-C₃)alkyl, S(O)₂-phenyl, 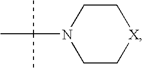
oxo-dihydrobenzimidazolyl, pyrazinyl, C(O)NH₂, C(O)NH-phenyl, C(O)-furanyl, C(O)NH(C₁-C₃)alkyl,
(C₁-C₃)alkyl optionally substituted with up to two substituents independently selected from OH, halo, (C₁-C₃)alkoxy, 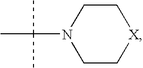
pyrrolidinyl, C(O)-pyrrolidinyl, 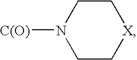
and N[(C₁-C₃)alkyl]₂,
phenyl optionally substituted with up to two substituents independently selected from (C₁-C₃)alkyl, (C₁-C₃)alkoxy, halo, CF₃, and CN, and
pyridyl optionally substituted with (C₁-C₃)alkyl, CF₃, and CN, and
pyrrolidinyl optionally substituted with up to two substituents independently selected from N[(C₁-C₄)alkyl]₂, C(O)NH₂, pyridyl, and
(C₁-C₃)alkyl optionally substituted with up to two substituents independently selected from (C₁-C₃)alkoxy, and pyrrolidinyl;
R⁶ is selected from H,
(C₁-C₄)alkyl optionally substituted with up to two substituents independently selected from OH, halo, (C₁-C₄)alkoxy, NHC(O)(C₁-C₃)alkyl, S—(C₁-C₃)alkyl, benzimidazolyl, thienyl, 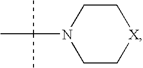
N[(C₁-C₄)alkyl]₂ where each alkyl is independently optionally substituted with up to two substituents independently selected from OH, (C₁-C₃)alkoxy, halo, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
indolyl optionally substituted up to two times with (C₁-C₃)alkyl,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, and halo,
indolyl optionally substituted up to two times with (C₁-C₃)alkyl,
pyrazolyl optionally substituted with up to two substituents independently selected from (C₁-C₄)alkyl, (C₃-C₆)cycloalkyl, and
phenyl optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl optionally substituted up to two times with (C₁-C₄)alkyl,
thiadiazolyl optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 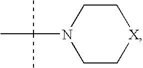
(C₁-C₄)alkoxy, O-pyridyl optionally substituted with C(O)NH(C₁-C₄)alkyl,
(C₁-C₄)alkyl optionally substituted with up to two substituents independently selected from pyridyl, OH, halo, and phenyl, and 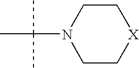
optionally substituted with up to two substituents independently selected from (C₁-C₃)alkyl, and (C₁-C₄)alkoxy,
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R⁷ is selected from (C₁-C₃)alkoxy, pyrrolidinyl, tetrahydropyranyl,
pyridyl optionally substituted with up to two substituents independently selected from (C₁-C₄)alkyl and (C₁-C₃)alkoxy,
pyranyl optionally substituted with up to two substituents independently selected from (C₁-C₄)alkyl and (C₁-C₃)alkoxy,
piperidinyl optionally substituted with up to two substituents independently selected from (C₁-C₃)alkyl, and (C₁-C₃)alkoxy, and
phenyl optionally substituted with up to two substituents independently selected from (C₁-C₃)alkoxy, and (C₁-C₃)alkyl; and
X is selected from O, S, CH₂ and NH;
with the proviso that when R¹ is F or Cl, then R⁴ must be H, and when R⁴ is F or Cl, then R¹ must be H;
or a pharmaceutically acceptable salt thereof.

3. A composition comprising a carrier and a compound of Formula I 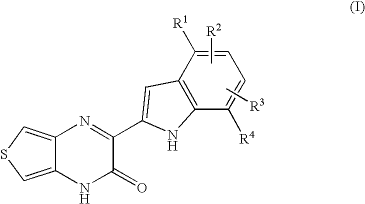
wherein
R¹ is selected from H, F, and Cl;
R² is selected from H, OH, CN, halo, C(O)R⁵, thienyl, pyrimidinyl, oxazolyl, furanyl,
(C₁-C₃)alkyl, (C₂-C₆)alkenyl and (C₂-C₆)alkynyl, each optionally substituted with up to two substituents selected from OH, halo, and (C₁-C₃)alkoxy optionally substituted with (C₁-C₃)alkoxy,
(C₁-C₆)alkoxy optionally substituted with (C₁-C₃)alkyl, (C₁-C₃)alkoxy, pyrrolidinyl, 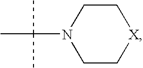
and N[(C₁-C₃)alkyl]₂ where each alkyl group is independently optionally substituted with a substituent selected from (C₁-C₃)alkyl, (C₁-C₃)alkoxy OH, halo, 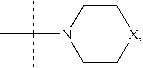
and phenyl,
N[(C₁-C₄)alkyl]₂ where each alkyl group is independently optionally substituted with up to two substituents independently selected from OH, (C₁-C₃)alkyl, halo, (C₁-C₃)alkoxy, and phenyl,
pyridyl optionally substituted with up to two substituents independently selected from (C₁-C₃)alkyl, (C₁-C₃)alkoxy, and halo,
phenyl optionally substituted with up to two substituents independently selected from (C₁-C₃)alkoxy, CN, halo, 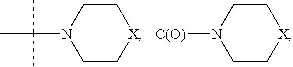
C(O)N[(C₁-C₃)alkyl]₂ where each alkyl is optionally substituted with (C₁-C₃)alkoxy, and
pyrrolidinyl optionally substituted with N[(C₁-C₃)alkyl]₂;
R³ is selected from H, halo, (C₁-C₃)alkyl, and (C₁-C₃)alkoxy;
R⁴ is selected from H, F, and Cl;
R⁵ is selected from OH, NHR⁶,
N[(C₁-C₃)alkyl]R⁷ where said alkyl is optionally substituted with up to one substituent selected from (C₁-C₃)alkyl and (C₁-C₃)alkoxy,
N[(C₁-C₃)alkyl]₂ where each alkyl is optionally substituted with up to two substituents independently selected from CN, OH, (C₁-C₃)alkoxy,
N[(C₁-C₃)alkyl]₂, pyridyl, phenyl, S(O)₂(C₁-C₃)alkyl, tetrahydrofuryl, S(O)₂-phenyl, (C₃-C₆)cycloalkyl, and
furyl optionally substituted with (C₁-C₃)alkyl,
N[(C₃-C₆)cycloalkyl](C₁-C₃)alkyl where said alkyl is substituted with up to two substituents independently selected from (C₁-C₃)alkoxy, OH, CN, N[(C₁-C₄)alkyl]₂, S(O)₂-phenyl, S(O)₂(C₁-C₃)alkyl, phenyl, furyl, tetrahydrofuryl, (C₅-C₆)cycloalkyl, and pyridyl, 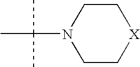
optionally substituted with up to two substituents independently selected from N[(C₁-C₃)alkyl]₂, C(O)(C₁-C₃)alkyl, pyrrolidinyl, S(O)₂(C₁-C₃)alkyl, S(O)₂-phenyl, 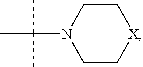
oxo-dihydrobenzimidazolyl, pyrazinyl, C(O)NH₂, C(O)NH-phenyl, C(O)-furanyl, C(O)NH(C₁-C₃)alkyl,
(C₁-C₃)alkyl optionally substituted with up to two substituents independently selected from OH, halo, (C₁-C₃)alkoxy, 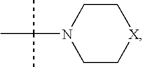
pyrrolidinyl, C(O)-pyrrolidinyl, 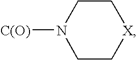
and N[(C₁-C₃)alkyl]₂,
phenyl optionally substituted with up to two substituents independently selected from (C₁-C₃)alkyl, (C₁-C₃)alkoxy, halo, CF₃, and CN, and
pyridyl optionally substituted with (C₁-C₃)alkyl, CF₃, and CN, and
pyrrolidinyl optionally substituted with up to two substituents independently selected from N[(C₁-C₄)alkyl]₂, C(O)NH₂, pyridyl, and
(C₁-C₃)alkyl optionally substituted with up to two substituents independently selected from (C₁-C₃)alkoxy, and pyrrolidinyl;
R⁶ is selected from H,
(C₁-C₄)alkyl optionally substituted with up to two substituents independently selected from OH, halo, (C₁-C₄)alkoxy, NHC(O)(C₁-C₃)alkyl, S—(C₁-C₃)alkyl, benzimidazolyl, thienyl, 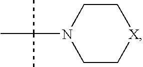
N[(C₁-C₄)alkyl]₂ where each alkyl is independently optionally substituted with up to two substituents independently selected from OH, (C₁-C₃)alkoxy, halo, and phenyl,
phenyl optionally substituted with up to two substituents independently selected from (C₁-C₃)alkyl, (C₁-C₃)alkoxy, CN, halo, CF₃, S(O)₂(C₁-C₃)alkyl, S(O)₂phenyl, and S(O)₂NH₂,
pyridyl optionally substituted up to two times with CF₃,
indolyl optionally substituted up to two times with (C₁-C₃)alkyl,
imidazolyl optionally substituted up to two times with (C₁-C₃)alkyl,
furyl optionally substituted up to two times with (C₁-C₄)alkyl, and
pyrrolidinyl optionally substituted with up to two substituents independently selected from (C₁-C₄)alkoxy, (O), and
(C₁-C₄)alkyl optionally substituted with up to two substituents independently selected from OH, (C₁-C₃)alkoxy, and halo,
indolyl optionally substituted up to two times with (C₁-C₃)alkyl,
pyrazolyl optionally substituted with up to two substituents independently selected from (C₁-C₄)alkyl, (C₃-C₆)cycloalkyl, and
phenyl optionally substituted with up to two substituents independently selected from (C₁-C₄)alkoxy, (C₁-C₄)alkyl, halo, CF₃, and CN,
benzothiazolyl optionally substituted up to two times with (C₁-C₄)alkyl,
thiazolyl optionally substituted up to two times with (C₁-C₄)alkyl,
thiadiazolyl optionally substituted with up to two substituents independently selected from CF₃, (C₃-C₆)cycloalkyl, and (C₁-C₆)alkyl,
phenyl optionally substituted with up to two substituents independently selected from CN, halo, CF₃, N[(C₁-C₄)alkyl]₂, indolyl, 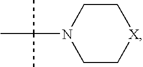
(C₁-C₄)alkoxy, O-pyridyl optionally substituted with C(O)NH(C₁-C₄)alkyl,
(C₁-C₄)alkyl optionally substituted with up to two substituents independently selected from pyridyl, OH, halo, and phenyl, and 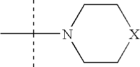
optionally substituted with up to two substituents independently selected from (C₁-C₃)alkyl, and (C₁-C₄)alkoxy,
pyridyl optionally substituted with phenoxy where said phenoxy is optionally substituted with up to two substituents independently selected from (C₁-C₄)alkyl and (C₁-C₄)alkoxy, and
indazolyl optionally substituted up to two times with (C₁-C₄)alkyl;
R⁷ is selected from (C₁-C₃)alkoxy, pyrrolidinyl, tetrahydropyranyl,
pyridyl optionally substituted with up to two substituents independently selected from (C₁-C₄)alkyl and (C₁-C₃)alkoxy,
pyranyl optionally substituted with up to two substituents independently selected from (C₁-C₄)alkyl and (C₁-C₃)alkoxy,
piperidinyl optionally substituted with up to two substituents independently selected from (C₁-C₃)alkyl, and (C₁-C₃)alkoxy, and
phenyl optionally substituted with up to two substituents independently selected from (C₁-C₃)alkoxy, and (C₁-C₃)alkyl; and
X is selected from O, S, CH₂ and NH;
with the proviso that when R¹ is F or Cl, then R⁴ must be H, and when R⁴ is F or Cl, then R¹ must be H;
or a pharmaceutically acceptable salt thereof.
